SEQUENCE LISTING (129 sequences)

SEQ ID NO: 1 - protein, 9 aa, Human papillomavirus 16
ISEYRHYCY

SEQ ID NO: 2 - protein, 10 aa, Human papillomavirus 16
HGDTPTLHEY

SEQ ID NO: 3 - protein, 11 aa, Human papillomavirus 16
TIHDIILQCVY

SEQ ID NO: 4 - protein, 11 aa, Human papillomavirus 16
YGVCDKCLKFY

SEQ ID NO: 5 - protein, 10 aa, Human papillomavirus 16
EYRYYCYSLY

SEQ ID NO: 6 - protein, 10 aa, Human papillomavirus 16
DLQPETTDLY

SEQ ID NO: 7 - protein, 9 aa, Human papillomavirus 16
TTDLYCYEQ

SEQ ID NO: 8 - protein, 11 aa, Human papillomavirus 16
SKISEYRHYCY

SEQ ID NO: 9 - protein, 11 aa, Human papillomavirus 16
SEYRHYCYSLY

SEQ ID NO: 10 - protein, 8 aa, Human papillomavirus 16
ETTDLYCY

SEQ ID NO: 11 - protein, 9 aa, Human papillomavirus 16
RLCVQSTHV

SEQ ID NO: 12 - protein, 11 aa, Human papillomavirus 16
LMGTLGIVCPI

SEQ ID NO: 13 - protein, 8 aa, Human papillomavirus 16
YMLDLQPE

SEQ ID NO: 14 - protein, 10 aa, Human papillomavirus 16
MGTLGIVCPI

SEQ ID NO: 15 - protein, 9 aa, Human papillomavirus 16
ELQTTIHEI

SEQ ID NO: 16 - protein, 11 aa, Human papillomavirus 16
TTIHEIILECV

SEQ ID NO: 17 - protein, 11 aa, Human papillomavirus 16
TTIHEIRLECV

SEQ ID NO: 18 - protein, 10 aa, Human papillomavirus 16
TIHEIILECV

SEQ ID NO: 19 - protein, 10 aa, Human papillomavirus 16
TIHEIRLECV

SEQ ID NO: 20 - protein, 10 aa, Human papillomavirus 16
SEYRYYCYSL

SEQ ID NO: 21 - protein, 10 aa, Human papillomavirus 16
SEYRYYCYSV

SEQ ID NO: 22 - protein, 8 aa, Human papillomavirus 16
YRYYCYSV

SEQ ID NO: 23 - protein, 10 aa, Human papillomavirus 16
RYYCYSLYGT

SEQ ID NO: 24 - protein, 10 aa, Human papillomavirus 16
LLMGTLGIVC

SEQ ID NO: 25 - protein, 11 aa, Human papillomavirus 16
LLMGTLGIVCP

SEQ ID NO: 26 - protein, 9 aa, Human papillomavirus 16
FQDPQERPI

SEQ ID NO: 27 - protein, 11 aa, Human papillomavirus 16
FQDPQERPIKL

SEQ ID NO: 28 - protein, 11 aa, Human papillomavirus 16
KLPQLCTELQT

SEQ ID NO: 29 - protein, 9 aa, Human papillomavirus 16
ELQTTIHDI

SEQ ID NO: 30 - protein, 11 aa, Human papillomavirus 16
TTIHDIILECV

SEQ ID NO: 31 - protein, 8 aa, Human papillomavirus 16
YRYYCYSL

SEQ ID NO: 32 - protein, 11 aa, Human papillomavirus 16
TLHEYMLDLQP

SEQ ID NO: 33 - protein, 11 aa, Human papillomavirus 16
IRTLEDLLMGT

SEQ ID NO: 34 - protein, 10 aa, Human papillomavirus 16
CVYCKQQLLR

SEQ ID NO: 35 - protein, 9 aa, Human papillomavirus 16
IILECVYCK

SEQ ID NO: 36 - protein, 9 aa, Human papillomavirus 16
HLDKKQRFH

SEQ ID NO: 37 - protein, 9 aa, Human papillomavirus 16
IVCPICSQK

SEQ ID NO: 38 - protein, 11 aa, Human papillomavirus 16
HEIILECVYCK

SEQ ID NO: 39 - protein, 11 aa, Human papillomavirus 16
FAFRDLCIVYR

SEQ ID NO: 40 - protein, 10 aa, Human papillomavirus 16
AFRDLCIVYR

SEQ ID NO: 41 - protein, 9 aa, Human papillomavirus 16
IVYRDGNPY

SEQ ID NO: 42 - protein, 11 aa, Human papillomavirus 16
NPYAVCDKCLK

SEQ ID NO: 43 - protein, 10 aa, Human papillomavirus 16
AVCDKCLKFY

SEQ ID NO: 44 - protein, 11 aa, Human papillomavirus 16
AVCDKCLKFYS

SEQ ID NO: 45 - protein, 11 aa, Human papillomavirus 16
VCDKCLKFYSK

SEQ ID NO: 46 - protein, 10 aa, Human papillomavirus 16
GIVCPICSQK

SEQ ID NO: 47 - protein, 11 aa, Human papillomavirus 16
MFQDPQERPRK

SEQ ID NO: 48 - protein, 11 aa, Human papillomavirus 16
YAVCDKCLKFY

SEQ ID NO: 49 - protein, 8 aa, Human papillomavirus 16
AVCDKCLK

SEQ ID NO: 50 - protein, 9 aa, Human papillomavirus 16
KCLKFYSKI

SEQ ID NO: 51 - protein, 9 aa, Human papillomavirus 16
KFYSKISEY

SEQ ID NO: 52 - protein, 10 aa, Human papillomavirus 16
KFYSKISEYR

SEQ ID NO: 53 - protein, 11 aa, Human papillomavirus 16
RHYCYSVYGTT

SEQ ID NO: 54 - protein, 9 aa, Human papillomavirus 16
TTLEQQYNK

SEQ ID NO: 55 - protein, 11 aa, Human papillomavirus 16
DLLIRCINCQK

SEQ ID NO: 56 - protein, 9 aa, Human papillomavirus 16
LIRCINCQK

SEQ ID NO: 57 - protein, 11 aa, Human papillomavirus 16
RCINCQKPLCP

SEQ ID NO: 58 - protein, 10 aa, Human papillomavirus 16
KQRFHNIRGR

SEQ ID NO: 59 - protein, 11 aa, Human papillomavirus 16
LGIVCPICSQK

SEQ ID NO: 60 - protein, 8 aa, Human papillomavirus 16
VYCKQQLL

SEQ ID NO: 61 - protein, 9 aa, Human papillomavirus 16
RAHYNIVTF

SEQ ID NO: 62 - protein, 10 aa, Human papillomavirus 16
TFCCKCDSTL

SEQ ID NO: 63 - protein, 11 aa, Human papillomavirus 16
PYAVCDKCLKF

SEQ ID NO: 64 - protein, 11 aa, Human papillomavirus 16
SEYRHYCYSVY

SEQ ID NO: 65 - protein, 10 aa, Human papillomavirus 16
VYGTTLEQQY

SEQ ID NO: 66 - protein, 11 aa, Human papillomavirus 16
PDRAHYNIVTF

SEQ ID NO: 67 - protein, 10 aa, Human papillomavirus 16
DRAHYNIVTF

SEQ ID NO: 68 - protein, 9 aa, Human papillomavirus 16
HYNIVTFCC

SEQ ID NO: 69 - protein, 8 aa, Human papillomavirus 16
TFCCKCDF

SEQ ID NO: 70 - protein, 10 aa, Human papillomavirus 16
VYRDGNPYGV

SEQ ID NO: 71 - protein, 11 aa, Human papillomavirus 16
HYCYSLYGTTL

SEQ ID NO: 72 - protein, 8 aa, Human papillomavirus 16
YSLYGTTL

SEQ ID NO: 73 - protein, 11 aa, Human papillomavirus 16
HLDKKQRFHNI

SEQ ID NO: 74 - protein, 8 aa, Human papillomavirus 16
DKKQRFHN

SEQ ID NO: 75 - protein, 8 aa, Human papillomavirus 16
AHYNIVTF

SEQ ID NO: 76 - protein, 8 aa, Human papillomavirus 16
RPRKLPQL

SEQ ID NO: 77 - protein, 9 aa, Human papillomavirus 16
TPTLHEYML

SEQ ID NO: 78 - protein, 11 aa, Human papillomavirus 16
RPRKLPQLCTE

SEQ ID NO: 79 - protein, 9 aa, Human papillomavirus 16
LEQQYNKPL

SEQ ID NO: 80 - protein, 11 aa, Human papillomavirus 16
NIRGRWTGRCM

SEQ ID NO: 81 - protein, 10 aa, Human papillomavirus 16
EPDRAHYNIV

SEQ ID NO: 82 - protein, 10 aa, Human papillomavirus 16
RPRKLPQLCT

SEQ ID NO: 83 - protein, 8 aa, Human papillomavirus 16
KPLCDLLI

SEQ ID NO: 84 - protein, 10 aa, Human papillomavirus 16
RCMSCCRSSR

SEQ ID NO: 85 - protein, 10 aa, Human papillomavirus 16
KQQLLRREVY

SEQ ID NO: 86 - protein, 9 aa, Human papillomavirus 16
AFRDLCIVY

SEQ ID NO: 87 - protein, 9 aa, Human papillomavirus 16
LQPETTDLY

SEQ ID NO: 88 - protein, 10 aa, Human papillomavirus 16
GQAEPDRAHY

SEQ ID NO: 89 - protein, 9 aa, Human papillomavirus 16
QQLLRREVY

SEQ ID NO: 90 - protein, 11 aa, Human papillomavirus 16
SVYGTTLEQQY

SEQ ID NO: 91 - protein, 10 aa, Human papillomavirus 16
LQPETTDLYC

SEQ ID NO: 92 - protein, 11 aa, Human papillomavirus 16
LQPETTDLYCY

SEQ ID NO: 93 - protein, 10 aa, Human papillomavirus 16
FAFRDLCIVY

SEQ ID NO: 94 - protein, 11 aa, Human papillomavirus 16
LCIVYRDGNPY

SEQ ID NO: 95 - protein, 9 aa, Human papillomavirus 16
YRHYCYSVY

SEQ ID NO: 96 - protein, 11 aa, Human papillomavirus 16
LLRREVYDFAF

SEQ ID NO: 97 - protein, 10 aa, Human papillomavirus 16
LRREVYDFAF

SEQ ID NO: 98 - protein, 11 aa, Human papillomavirus 16
IVYRDGNPYAV

SEQ ID NO: 99 - protein, 9 aa, Human papillomavirus 16
AVCDKCLKF

SEQ ID NO: 100 - protein, 11 aa, Human papillomavirus 16
CLKFYSKISEY

SEQ ID NO: 101 - protein, 8 aa, Human papillomavirus 16
FYSKISEY

SEQ ID NO: 102 - protein, 10 aa, Human papillomavirus 16
YSKISEYRHY

SEQ ID NO: 103 - protein, 9 aa, Human papillomavirus 16
SKISEYRHY

SEQ ID NO: 104 - protein, 8 aa, Human papillomavirus 16
SEYRHYCY

SEQ ID NO: 105 - protein, 10 aa, Human papillomavirus 16
QQYNKPLCDL

SEQ ID NO: 106 - protein, 9 aa, Human papillomavirus 16
CQKPLCPEE

SEQ ID NO: 107 - protein, 11 aa, Human papillomavirus 16
KQRHLDKKQRF

SEQ ID NO: 108 - protein, 11 aa, Human papillomavirus 16
KQRFHNIRGRW

SEQ ID NO: 109 - protein, 9 aa, Human papillomavirus 16
TLHEYMLDL

SEQ ID NO: 110 - protein, 9 aa, Human papillomavirus 16
GQAEPDRAH

SEQ ID NO: 111 - protein, 9 aa, Human papillomavirus 16
LLMGTLGIV

SEQ ID NO: 112 - protein, 10 aa, Human papillomavirus 16
TTDLYCYEQL

SEQ ID NO: 113 - protein, 8 aa, Human papillomavirus 16
SEYRHYCY

SEQ ID NO: 114 - protein, 10 aa, Human papillomavirus 16
IHEIILECVY

SEQ ID NO: 115 - protein, 10 aa, Human papillomavirus 16
GVCDKCLKFY

SEQ ID NO: 116 - protein, 10 aa, Human papillomavirus 16
GTTLEQQYNK

SEQ ID NO: 117 - protein, 8 aa, Human papillomavirus 16
RHYCYSLY

SEQ ID NO: 118 - protein, 10 aa, Human papillomavirus 16
GVCDKCLKFY

SEQ ID NO: 119 - protein, 8 aa, Human papillomavirus 16
HYNIVTFC

SEQ ID NO: 120 - protein, 10 aa, Human papillomavirus 16
TFCCKCDFTL

SEQ ID NO: 121 - protein, 8 aa, Human papillomavirus 16
RPGKLPQL

SEQ ID NO: 122 - protein, 10 aa, Human papillomavirus 16
RPRKLPDLCT

SEQ ID NO: 123 - protein, 9 aa, Human papillomavirus 16
CPDEKQRHL

SEQ ID NO: 124 - protein, 10 aa, Human papillomavirus 16
LPQLCTELQT

SEQ ID NO: 125 - protein, 8 aa, Human papillomavirus 16
AHYNIVTF

SEQ ID NO: 126 - protein, 8 aa, Human papillomavirus 16
KISEYRHY

SEQ ID NO: 127 - protein, 9 aa, Human papillomavirus 16
RGRWTGRCM

SEQ ID NO: 128 - protein, 11 aa, Human papillomavirus 16
AGQAEPDRAHY

SEQ ID NO: 129 - protein, 8 aa, Human papillomavirus 16
FYSKISEY

(19) United States
(12) Patent Application Publication
(10) Pub. No.: US 2026/0240975 A1
(43) Pub. Date: Aug. 20, 2026

(54) VALIDATED HPV16-DERIVED STIMULATION PEPTIDES

(71) Applicant: Deutsches Krebsforschungszentrum Stiftung des öffentlichen Rechts, Heidelberg (DE)

(72) Inventors: Angelika Beate RIEMER, Heidelberg (DE); Maria BONSACK, Heidelberg (DE); Jonas FÖRSTER, Heidelberg (DE); Jonas BECKER, Heidelberg (DE); Kathrin WELLACH, Heidelberg (DE)

(73) Assignee: Deutsches Krebsforschungszentrum Stiftung des öffentlichen Rechts, Heidelberg (DE)

(21) Appl. No.: 19/160,552

(22) PCT Filed: Mar. 1, 2024
(86) PCT No.: PCT/EP2024/055432

§ 371 Date: Aug. 28, 2025

(30) Foreign Application Priority Data
Mar. 2, 2023 (EP) 23159742.8

(51) Int. Cl. A61K 39/12; A61K 39/00; C07K 14/005; C07K 14/705

(52) U.S. Cl. CPC A61K 39/12; A61K 39/0011; C07K 14/005; C07K 14/70539; C12N 2710/20022; C12N 2710/20034

(57) ABSTRACT

The present invention relates to an in vitro method for producing an immunoreactive agent against human cancer cells infected with HPV16-related virus, said method comprising expressing an at least partial nucleic acid sequence encoding an immunoreactive agent obtained from an immune cell stimulated by a complex comprising a human leukocyte antigen (HLA) and a stimulation peptide (stimulation complex), wherein said stimulation peptide (i) consists of an amino acid sequence selected from SEQ ID NOs: 1 and 2 and said HLA is from HLA supertype HLA-A01; (ii) consists of an amino acid sequence of SEQ ID NO: 11 and said HLA is from HLA supertype HLA-A02, (iii) consists of an amino acid sequence selected from SEQ ID NOs:34 to 37 and said HLA is from HLA supertype HLA-A03/A11; (iv) consists of an amino acid sequence selected from SEQ ID NOs:60 and bland said HLA is from HLA supertype HLA-A24; (v) consists of an amino acid sequence selected from SEQ ID NOs:61, 76, and 77 and said HLA is from HLA supertype HLA-B07; or (vi) consists of an amino acid sequence selected from SEQ ID NOs:61 and 85 to 92 and said HLA is from HLA supertype HLA-B15; and wherein an HPV16-derived peptide consisting of the same amino acid sequence as said stimulation peptide has been verified to be presented by human HPV16-positive cancer cells, and to methods and uses related thereto.

The present invention relates to an in vitro method for producing an immunoreactive agent against human cancer cells infected with HPV16-related virus, said method comprising expressing an at least partial nucleic acid sequence encoding an immunoreactive agent obtained from an immune cell stimulated by a complex comprising a human leukocyte antigen (HLA) and a stimulation peptide (stimulation complex), wherein said stimulation peptide (i) consists of an amino acid sequence selected from SEQ ID NOs:1 and 2 and said HLA is from HLA supertype HLA-A01; (ii) consists of an amino acid sequence of SEQ ID NO: 11 and said HLA is from HLA supertype HLA-A02, (iii) consists of an amino acid sequence selected from SEQ ID NOs:34 to 37 and said HLA is from HLA supertype HLA-A03/A11; (iv) consists of an amino acid sequence selected from SEQ ID NOs:60 and 61 and said HLA is from HLA supertype HLA-A24; (v) consists of an amino acid sequence selected from SEQ ID NOs:61, 76, and 77 and said HLA is from HLA supertype HLA-B07; or (vi) consists of an amino acid sequence selected from SEQ ID NOs:61 and 85 to 92 and said HLA is from HLA supertype HLA-B15; and wherein an HPV16-derived peptide consisting of the same amino acid sequence as said stimulation peptide has been verified to be presented by human HPV16-positive cancer cells, and to methods and uses related thereto.

At least 20% of human malignancies are caused by consequences of persistent infections. Cancers caused by infectious agents are attractive targets for cancer vaccination approaches, as they provide the opportunity to target antigens that are immunological non-self. Vaccination can be prophylactic, inducing an immune response that prevents infection in the first place, or therapeutic, stimulating the immune system into eradicating established disease. Prophylactic immunization against certain high-risk human papillomavirus (HPV) types or against hepatitis B has become the paradigm for cancer immunoprevention.

As for therapeutic vaccination, HPV-associated neoplasia could again emerge as the model case: induction and maintenance of the malignant phenotype depends on two viral oncoproteins, E6 and E7. It is known from studies on spontaneously regressing HPV-induced lesions, and from HPV prevalence in immunosuppressed and HIV-infected patients, that cell-mediated immune responses are crucial in clearing established HPV infection. Various forms of vaccines, including peptide vaccines, targeting T cells to E6 and E7 have already been explored.

Candidate peptides proposed for vaccination against HPV are abundant in the literature, e.g. from Bonsack et al. (2019), Cancer Immunol Res; 7(5):719; Blatnik (2018), Proteomics 18:1700390, DOI: 10.1002/pmic.201700390; Krishna et al. (2018) Cancer Res.2018; 78(21):6159; Tsang et al. (2017), Vaccine 35(19):2605; Kast et al., J Immunol 1994; 152(8):3904; Ressing et al., J Immunol 1995; 154(11):5934; Bourgault Villada et al., Clin Exp Immunol 2010; 159(1):45; Mizuuchi et al., Exp Mol Pathol 2012; 92(1):185); Hara et al., Int J Oncol 2005; 27(5):1371; Jang et al., Cancer 2012; 118(8):2173, and Riemer et al., J Biol Chem 2010; 285(38):29608. Regarding peptide vaccines, it had, however, to be realized over the years that the processes leading to an effective T cell response are complex and require more than potential binding of a peptide to an MHC. In the case of MHC class I presentation, a precursor peptide must be cleaved by the proteasome of a presenting cell to provide the peptide in the first place; moreover, T cell activation may require cross-presentation of the peptide on MHC class I by APCs, which may cleave precursor peptides differently from non-APC cells. Moreover, there must be a T cell with the propensity to recognize the presented peptide, which crucially depends on the reservoir of T cell receptors available (cf. e.g. Becker & Riemer (2022), Frontiers Immunol, doi: 10.3389/fimmu.2022.883989; Habib et al. (2022), Cells 6; 11(3):421. doi: 10.3390/cells11030421.). Also, in cancer cells, immune evasion mechanisms may prevent proper presentation of HPV-derived peptides (Steinbach & Riemer (2018), Int J Cancer 142:224). In conclusion, experimental binding of a candidate peptide to MHC class I in itself, unfortunately, does not make this candidate peptide already a plausible candidate for a vaccine.

In view of the above, it is clear that verifying candidate HPV peptides as indeed suitable for vaccination is by far a non-routine task: in presentation assays, the amounts of peptide obtainable are minute, and for many peptides, fragmentation patterns in mass spectrometry are complex. It is, thus, that in a study of 2010 (Riemer et al. (2010), loc.cit.), of 21 peptides predicted to be presented via HLA-A*0201, only one could be confirmed to be presented. After eight years of method development, 17 peptides could be confirmed from a predicted group of 121 potential HLA-A2 binding peptides (Blatnik et al. (2018), loc. cit.). Notably, cysteine-containing peptides were excluded altogether, as is usual in the art, since they are prone to intra- and intermolecular reactions, which complicate analysis and further reduce the amounts of defined and detectable ions available for MS analysis. If a HPV peptide cannot be identified as suitable by the in vitro methods described above, the only remaining alternative is in vivo testing, which requires extended animal and/or human testing by vaccination.

Also, for successful therapeutic vaccination against HPV, it is not only necessary that the vaccinated peptide is immunogenic, e.g. by activating T cells when presented via MHC molecules, but the peptide also has to be presented by target cells, i.e. HPV-infected cells. As noted in Riemer et al. ((2010), loc. cit.), it is essential for vaccination to define HPV-16 E6 and E7 T cell epitopes that are naturally processed and presented on the surface of virally altered cells. Only those HPV peptide/MHC class I complexes are capable of being recognized by cytolytic T lymphocytes to target destruction of transformed cells. Thus, success of a therapeutic HPV vaccine is dependent on accurate identification of HPV epitopes displayed on HPV-infected cells. Apart from direct proof of MHC presentation by MS analysis, investigating memory responses in healthy donors can serve as a surrogate test for presentation. If memory immune responses are present, the epitope in question must have been presented during a previous encounter with the virus. However, even proven natural immunogenicity is not sufficient to validate a peptide in the context of its restricting HLA complex as an actionable target. This is because APCs use a different proteasome, the immunoproteasome, compared to normal body cells, and furthermore, antigen processing of HPV16-infected/transformed cells is heavily modulated by the virus. Thus, a memory T cell response only proves that the peptide was presented by antigen-presenting cells (APCs), not that this epitope will still be presented on HPV-dependent tumor cells. Thus, not every possible HPV epitope is presented on HPV-positive target cells.

Thus, to be an actionable target, a HPV16-derived peptide must bind to an HLA molecule, be immunogenic, and must also be presented on the target cell, in particular an HPV16 infected or transformed cell.

There is, thus, a need in the art for improved means and methods for immunoreactive agents against cells transformed by HPV, such as HPV-positive cancer cells, and reagents related thereto, without the drawbacks as referred to above.

The technical problem underlying the present invention can be seen as the provision of means and methods for complying with the aforementioned needs. The technical problem is solved by the embodiments characterized in the claims and herein below.

In accordance, the present invention relates to a method for stimulating immune cells specifically binding to human host cells infected with an HPV16-related virus, said method comprising
(A) contacting a complex comprising a human leukocyte antigen (HLA) and a stimulation peptide (stimulation complex) with immune cells, and
(B) thereby stimulating immune cells specifically binding to HPV16-related virus-infected host cells,
wherein said stimulation peptide consists of an amino acid sequence selected from SEQ ID NOs:1 to 111, in a preferred embodiment selected from SEQ ID NOs: 1 to 129.

The method for stimulating immune cells, preferably, is an in vitro method, which may, preferably e.g. be performed on an isolated sample of a subject. The method may, preferably, however, also be performed in vivo, more preferably in a non-human experimental animal. The method may comprise further steps beyond those expressly specified and may be assisted or performed by automated equipment.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. In accordance, expressions relating to one item of a plurality, unless otherwise indicated, preferably relate to at least one such item, more preferably a plurality thereof, thus, e.g. identifying “a cell” relates to identifying at least one cell, preferably to identifying a multitude of cells.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, preferably, are in vitro methods. The method steps may, in principle, be performed in any arbitrary sequence deemed suitable by the skilled person, but preferably are performed in the indicated sequence; also, one or more, preferably all, of said steps may be assisted or performed by automated equipment. Moreover, the methods may comprise steps in addition to those explicitly mentioned above.

As used herein, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “fragment” of a biological macromolecule, preferably of a polynucleotide or polypeptide, is used herein in a wide sense relating to any sub-part, preferably subdomain, of the respective biological macromolecule comprising the indicated sequence, structure and/or function. Thus, the term includes sub-parts generated by actual fragmentation of a biological macromolecule, but also sub-parts derived from the respective biological macromolecule in an abstract manner, e.g. in silico. Thus, as used herein, an Fc or Fab fragment, but also e.g. a single-chain antibody, a bispecific antibody, and a nanobody may be referred to as fragments of an immunoglobulin.

Unless specifically indicated otherwise herein, the compounds specified, in particular the polynucleotides and (poly)peptides, may be comprised in larger structures, e.g. may be covalently or non-covalently linked to further sequences, carrier molecules, retardants, and other excipients. In particular, peptides and (poly)peptides as specified may be comprised in fusion polypeptides comprising further peptides, which may serve e.g. as a tag for purification and/or detection, as a linker, or to extend the in vivo half-life of a compound. The term “detectable tag” refers to a stretch of amino acids which are added to or introduced into the fusion polypeptide; preferably, the tag is added C- or N-terminally to the fusion polypeptide. Said stretch of amino acids preferably allows for detection of the polypeptide by an antibody which specifically recognizes the tag; or it preferably allows for forming a functional conformation, such as a chelator; or it preferably allows for visualization, e.g. in the case of fluorescent tags. Preferred detectable tags are the Myc-tag, FLAG-tag, 6-His-tag, HA-tag, GST-tag or a fluorescent protein tag, e.g. a GFP-tag. These tags are all well known in the art. Other further peptides preferably comprised in a fusion polypeptide comprise further amino acids or other modifications which may serve as mediators of secretion, as mediators of blood-brain-barrier passage, as cell-penetrating peptides, and/or as immune stimulants. Further polypeptides or peptides to which the polypeptides may be fused are signal and/or transport sequences, e.g. an IL-2 signal sequence, and linker sequences.

The term “polypeptide”, as used herein, refers to a molecule consisting of several, typically at least 20 amino acids that are covalently linked to each other by peptide bonds. Molecules consisting of less than 20 amino acids covalently linked by peptide bonds are usually considered to be “peptides”. The peptides referred to herein are disclosed as SEQ ID NOs:1 to 111, as well as in Table 1 herein below. As the skilled person understands from the disclosure herein, the stimulation peptides described herein are preferably used with the HLA supertypes indicated herein in Table 1. Thus, preferably used are the immunization peptide/HLA supertype combinations of Tables 1(i) to (vi).

The term “polynucleotide” is known to the skilled person. As used herein, the term includes nucleic acid molecules comprising or consisting of a nucleic acid sequence or nucleic acid sequences as specified herein and/or encoding at least one stimulation peptide. The polynucleotide of the present invention shall be provided, preferably, either as an isolated polynucleotide (i.e. isolated from its natural context) or in genetically modified form. The polynucleotide, preferably, is DNA, including cDNA, or is RNA. The term encompasses single as well as double stranded polynucleotides, as well as circularly closed and linear polynucleotides. Preferably, the polynucleotide is a chimeric molecule, i.e., preferably, comprises at least one nucleic acid sequence, preferably of at least 20 bp, more preferably at least 100 bp, heterologous to the residual nucleic acid sequence(s) or being an artificial nucleic acid sequence. Moreover, preferably, comprised are also chemically modified polynucleotides including naturally occurring modified polynucleotides such as glycosylated or methylated polynucleotides or artificial modified ones such as biotinylated polynucleotides.

The polynucleotide may be comprised in an expression construct. The term “expression construct”, as used herein, refers to a heterologous polynucleotide comprising the aforementioned polynucleotide as well as nucleic acid sequences required for expression of the polynucleotide. Typically, such additional nucleic acid sequences, which preferably are heterologous to the polynucleotide encoding the at least one stimulation peptide, may be promoter sequences, regulatory sequences and/or transcription termination sequences, such as terminators. Preferably, the expression construct is a eukaryotic expression construct, i.e. an expression construct comprising all elements required for expression, preferably inducible expression, in a eukaryotic host cell. Suitable expression control sequences are well known in the art and include in particular the CMV promoter or other constitutive promoters. However, inducible and/or cell-type specific promoters may be used as well.

The polynucleotide and/or the expression construct may be comprised in a vector. The term “vector”, as used herein, relates to any polynucleotide adapted for stably maintaining the polynucleotide and/or the expression construct as specified herein above in a host cell. Thus, the term vector preferably encompasses phage, plasmid, and viral vectors as well artificial chromosomes, such as bacterial or yeast artificial chromosomes. Preferably, the vector is a plasmid or a virus-derived vector, preferably a replication-incompetent viral vector. Moreover, the term also relates to targeting constructs which allow for random or site-directed integration of the targeting construct into genomic DNA of a host cell. Such target constructs, preferably, comprise DNA of sufficient length for either homologous or heterologous recombination. The vector encompassing the polynucleotide and/or the expression construct as specified herein above, preferably, further comprises at least one selectable marker for propagation and/or selection of a host cell. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerenes. Alternatively, a plasmid vector may be introduced by heat shock or electroporation techniques. Should the vector be a virus, it may be packaged in vitro using an appropriate packaging cell line prior to application to host cells. Preferably, the vector is a vertebrate vector, more preferably a mammalian vector, or a shuttle vector. Preferably, the vector is an expression vector and/or a gene transfer or targeting vector. Methods which are well known to those skilled in the art can be used to construct recombinant polynucleotides and vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Preferably, the vector is an AAV vector, or a lentiviral vector.

Methods for determining stimulation of immune cells are known in the art and are described elsewhere herein in particular in the Examples. Preferably, stimulation is significant stimulation compared to an untreated control and/or a vehicle control.

The terms “human papillomavirus 16-related virus” and “HPV16-related virus” relate to any virus, preferably infecting humans, encoding at least one amino acid sequence of SEQ ID NOs: 1 to 111. Preferably, the HPV16-related virus is a papillomavirus (PV), more preferably a human papillomavirus (HPV), still more preferably is selected from the list consisting of HPV16, HPV31, HPV33, HPV35, HPV52, HPV58, and HPV67, most preferably is HPV16.

The term “subject”, as used herein, relates to a vertebrate animal, preferably a mammal, more preferably a human. Preferably the subject comprises at least one HLA type as specified herein below in Table 1. Preferably, the subject is infected with at least one HPV16-related virus. Preferably, the subject is afflicted with at least one HPV16-related virus positive inappropriate cellular proliferation.

The term “inappropriate cellular proliferation” relates to an abnormal proliferation of body cells in a subject; as a consequence, an imbalance of cellular composition of a body tissue, of a body fluid and/or tumor formation may ensue. Inappropriate cellular proliferation may be induced by an infectious agent, preferably a virus, more preferably an HPV16-related virus. Preferably, inappropriate cellular proliferation is benign, i.e. preferably, does not threaten health or life of a subject. Preferred benign inappropriate cellular proliferations are warts, exophytic growing papillomas, condylomata, inverted papillomas, and pre-neoplastic HPV-induced lesions. Also preferably, the inappropriate cellular proliferation is pre-malignant or malignant, i.e. does at least potentially threaten health or life of a subject; thus, preferably, the inappropriate cellular proliferation is a pre-malignant or malignant inappropriate proliferation of a mucosa or a skin, in particular a mucosa, e.g. of the oropharynx, anogenital regions, and/or reproductive organs, e.g. cervical intraepithelial neoplasia (CIN) or cancer, in particular cervical cancer and/or head and neck cancer. The term “cancer”, as used herein, relates to a disease of an animal, including man, characterized by uncontrolled growth by a group of body cells (“cancer cells”). This uncontrolled growth may be accompanied by intrusion into and destruction of surrounding tissue and possibly spread of cancer cells to other locations in the body. Preferably, also included by the term cancer is a relapse. Thus, preferably, the cancer is a solid cancer, a metastasis, or a relapse thereof. Thus, eliciting an immune response preferably is cancer prevention and/or cancer treatment.

The term “stimulation peptide”, as used herein, relates to any peptide consisting of an amino acid sequence selected from SEQ ID NOs: 1 to 111. As the skilled person will understand from the Examples provided herein, the aforesaid peptides were verified to activate human T cells when presented to human T cells as an human leukocyte antigen (HLA) complex. Preferably, the stimulation peptide consists of an amino acid sequence selected from SEQ ID NOs:1, 2, 11, 34-37, 60, 61, 76, 77, and 85-92, which, as shown herein below in the Examples, were additionally verified to be presented by human cancer cells infected with a HPV-16-related virus. Thus, stimulation peptides consisting of an amino acid sequence selected from SEQ ID SEQ ID NOs:1, 2, 11, 34-37, 60, 61, 76, 77, and 85-92 were verified to be presented by human cancer cells infected with a HPV-16-related virus and were verified to activate human T cells when presented to human T cells as an HLA complex. As the skilled person will understand, a human cancer cells infected with a HPV-16-related virus may lose parts of the HPV genome; in accordance, as used herein, any cancer cell expressing at least the HPV16-related E6 and E7 polypeptides preferably is a human cancer cell infected with a HPV-16-related virus.

In a preferred embodiment, the immunization peptide consists of an amino acid sequence comprising at least one, preferably at least two, more preferably three, cysteine residue(s). Thus, the immunization peptide, in a preferred embodiment, is selected from the list consisting of SEQ ID NOs: 1, 3-5, 7-12, 14, 16-25, 28, 30, 31, 34, 35, 37-40, 42-46, 48-50, 53, 55-57, 59, 60, 62-64, 68, 69, 71, 78, 80, 82-84, 86, 91-95, 99, 100, 104-106, 112-115, 117-120, 122-124, and 127, more preferably consisting of SEQ ID NOs: 4, 34, 35, 37, 38, 42-46, 48, 49, 55-57, 59, 62, 63, 68, 69, 84, 99, 106, 115, 118, and 120, even more preferably consisting of SEQ ID NOs:57, 62, 69, 84, and 120. In an also preferred embodiment, the immunization peptide consists of an amino acid sequence comprising at least one cysteine residue and is presented via HLA-A01, preferably is selected from the list consisting of SEQ ID NOs:1, 3-5, 7-10, and 112-115; in a further preferred embodiment, the immunization peptide consists of an amino acid sequence comprising at least one cysteine residue and is presented via HLA-A02, preferably is selected from the list consisting of SEQ ID NOs:11, 12, 14, 16-25, 28, 30, 31; in a further preferred embodiment, the immunization peptide consists of an amino acid sequence comprising at least one cysteine residue and is presented via HLA-A03, preferably is selected from the list consisting of SEQ ID NOs:34, 37, 43, 46, 49, 50, 53, 56, 57, 117, and 118; in a further preferred embodiment, the immunization peptide consists of an amino acid sequence comprising at least one cysteine residue and is presented via HLA-A11, preferably is selected from the list consisting of SEQ ID NOs:39, 40, 34, 35, 37, 38, and 42-45; in a further preferred embodiment, the immunization peptide consists of an amino acid sequence comprising at least one cysteine residue and is presented via HLA-A24, preferably is selected from the list consisting of SEQ ID NOs:60, 62-64, 68, 69, 71, and 119; in a further preferred embodiment, the immunization peptide consists of an amino acid sequence comprising at least one cysteine residue and is presented via HLA-B07, preferably is selected from the list consisting of SEQ ID NOs:12, 78, 80, 82-84, and 122-124; in a further preferred embodiment, the immunization peptide consists of an amino acid sequence comprising at least one cysteine residue and is presented via HLA-B15, preferably is selected from the list consisting of SEQ ID NOs:9, 43, 64, 80, 86, 91-95, 99, 100, 104-106, and 127. In a further preferred embodiment, the immunization peptide consists of an amino acid sequence comprising at least one cysteine residue and is a peptide verified herein to be presented on cancer cells infected with HPV16, i.e. preferably is a selected from the list consisting of SEQ ID NOs:1, 11, 34, 35, 37, 60, 86, 91, and 92.

The term “human leukocyte antigen”, which may also be referred to as “HLA”, is in principle understood by the skilled person to relate to a family of cellular surface proteins presenting peptides to immune cells. HLAs are also known as major histocompatibility complexes, HLAs A, B, and C corresponding to MHC class I, and HLAs DP, DM, DQ, and DR corresponding to MHC class II. As will be understood from Table 1 provided herein below, the stimulation peptides referred to herein bind to HLAs A, B, and C, so the HLA preferably is HLA A, B, or C, i.e. is MHC class I. The term HLA is used herein in a broad sense as relating to any HLA molecule or fragment thereof having the activity of presenting a stimulation peptide to immune cells, preferably such that a cognate T cell becomes activated by the complex comprising the HLA and the stimulation peptide. Thus, a HLA may e.g. be a soluble HLA or an HLA-oligomer, the term “HLA-oligomer” relating to an oligomeric form of a soluble HLA. In the HLA-oligomer, a soluble, non-membrane bound, form of the HLA is used; moreover, the HLA is used as an oligomer, the term “oligomer” preferably relating to an at least dimeric form, preferably at least trimeric form; preferably, the oligomer comprises of from two to 8 HLA molecules, more preferably of from three to 6 HLA molecules, most preferably four HLA molecules. Thus, the HLA oligomer preferably is an HLA-tetramer. Preferably, the HLA molecules in the HLA-oligomer are connected via biotin-streptavidin affinity pairs. Preferably, the HLA-oligomer comprises a detectable label, e.g. a fluorescent marker. Appropriate methods of providing HLA-oligomers are known in the art, e.g. from Altman et al. (1996), Science 274:94. Further, HLA-oligomers are commercially available.

The term “stimulation complex” as referred to herein, relates to any complex comprising a human leukocyte antigen (HLA) and a stimulation peptide, both as specified herein above. The stimulation complex may be provided as a soluble stimulation complex, which may optionally comprise further components, e.g. co-stimulatory molecules, or may be comprised on a host cell, such as an antigen presenting cell loaded in vitro with a stimulation peptide; also, synthetic APCs or scaffolds mimicking the same may be used. Also, the immune complex may be produced on a host cell by contacting said host cell with an administered stimulation peptide. Since the stimulation peptide preferably is presented via MHC class I, the host cells in such cases may in principle be any host cells expressing MHC class I. Preferred stimulation complexes are those comprising the stimulation peptide/HLA combinations as shown in Table 1, preferably those designated as Complex ID NOs: 1, 2, 11, 34-38, 61, 62, 78-80, and 89-97.

The term, “immune cell”, as used herein, includes any and all cells having the capacity of recognizing, i.e. preferably specifically binding to, a stimulation complex as specified herein elsewhere. Thus, immune cells preferably are T cells expressing a T cell receptor specifically binding to the immune complex and/or are B cells specifically binding to the immune complex, said T cells preferably are CD8+ T cells, more preferably cytotoxic CD8+ T cells.

The term “stimulating immune cells” is understood by the skilled person and preferably includes an increase of at least one feature of an activated immune cell. Thus, the term includes initiating a de novo response as well as activating or enhancing an existing immune response. Stimulating immune cells preferably is eliciting or enhancing a T cell response, most preferably a CD8+ T cell response to a stimulation complex; also preferably, stimulating immune cells is eliciting or enhancing a B cell response to a stimulation complex, in particular production of antibodies against said immune complex. In accordance, stimulating immune cells preferably comprises increasing in the subject the number of immune cells specifically recognizing said stimulation complex, wherein; preferably said immune cells are T cells, preferably CD8+ T cells, more preferably cytotoxic CD8+ T cells. Thus, the stimulation peptide preferably is presented via a major histocompatibility complex (MHC) class I molecule, preferably on a nucleated cell, more preferably on an antigen presenting cell and/or on a nucleated cell infected with a HPV16-related virus, e.g. cells of an HPV16-related virus-positive inappropriate cellular proliferation, in particular a HPV-related lesion or HPV-related cancer as specified herein above. Thus, stimulating immune cells preferably comprises eliciting an immune response to a HPV16-related virus-positive inappropriate cellular proliferation. Preferably, stimulating immune cells comprises presentation of the stimulation peptide on at least one HLA type belonging to one of HLA supertypes A1, A2, A3/A11, A24, B7, and B15 (Sidney et al. (2008), BMC Immunology 9 Art. No. 1, doi.org/10.1186/1471-2172-9-1), preferably as allocated to specific stimulation peptides in Table 1. Preferably, stimulating immune cells comprises administering an agent providing a stimulation peptide as specified herein above to a subject, wherein said subject preferably is a non-human animal and preferably is sacrificed after said stimulating, preferably after steps (C) or (D) as specified herein below. Stimulating immune cells may, however, also relate to in vitro activating and/or enriching immune cells recognizing and/or binding the stimulation complex, optionally followed.

As used herein, the term “host cell” relates to any cell capable of receiving and presenting said stimulation peptide via an HLA. Preferably, the host cell is a eukaryotic cell, preferably an animal cell, e.g. an insect cell or a mammalian cell. More preferably, the host cell is a cell of a livestock, companion, or laboratory animal. Most preferably, the host cell is a human cell.

Preferably, the method is a method for stimulating and identifying immune cells specifically binding to a stimulation complex, preferably to human host cells infected with an HPV16-related virus; in such case, the method preferably further comprises (C) identifying stimulated immune cells stimulated in step (B).

The term “identifying” immune cells is used herein in a broad sense relating to any method making immune cells recognizing a stimulation complex discernible over immune cells not recognizing said stimulation complex. Appropriate methods are in principle known to the skilled person and preferably include contacting immune stimulated as specified herein above with a derivative of the stimulation complex carrying a detectable label, wherein the term “derivative of the stimulation complex carrying a detectable label” relates to the stimulating complex, preferably as used in step (B) as specified herein above, connected to, covalently or non-covalently, to a detectable label. The detectable label may be any label deemed appropriate by the skilled person. Preferably, the “detectable label” is a label detectable by optical means, which are in principle known in the art. More preferably, the detectable label is an optically detectable polypeptide, more preferably a fluorescent group, e.g. a fluorescent dye.

Also preferably, the method is a method for stimulating and enriching immune cells specifically binding to a stimulation complex, preferably to human host cells infected with an HPV16-related virus, and wherein said method further comprises step (D) enriching stimulated immune cells stimulated in step (B) and optionally identified in step (C).

The term “enriching”, as used herein, relates to any method increasing the number of immune cells recognizing the immune complex relative to those that do not in a preparation. Appropriate methods are known in the art and include in particular methods comprising contacting immune stimulated with a derivative of the stimulation complex carrying a detectable label as specified herein above and enriching immune cells binding to said stimulation complex by said detectable label. Thus, the term enriching is used herein in its conventional meaning. In the context of enriching a cell type, the term preferably relates to increasing the fraction of the enriched cell type over other cell types present in a composition. Methods of enriching a particular cell type usually comprise labelling a cell type of interest and applying an appropriate enrichment method, e.g. FACS and/or affinity based binding to a solid surface. As the skilled person understands, enriching may also comprise labeling of an undesired cell population and removal of such labeled cells. Using analogous methods, furthermore T cells expressing CD3, CD8, CD137, and/or IFN-7 in addition to a T cell receptor recognizing a stimulation peptide may be enriched.

Advantageously, it was found in the work underlying the present invention that HLA presentation of HPV16-derived peptides by HPV16-positive cells can be evaluated by mass spectrometry as disclosed in the Examples, providing experimental proof that peptides are in fact presented to the immune system on the intended target cells. Also, immunogenicity assays as shown in the Examples, performed on peripheral blood lymphocytes (PBMCs) of various donors can be used to provide proof that respective peptides are indeed active in vivo in inducing immune responses. Thus, in order to provide actionable target structures for T cell epitope-centered immunotherapies against HPV16-mediated malignancies (therapeutic vaccines, adoptive transfer of T cells with a transgenic TCR recognizing a validated HLA/peptide complex, or therapeutics based on antibodies recognizing the validated HLA/peptide complex (i.e. CAR-T cells or bispecific antibodies that bring a target cell and an effector cell into close proximity)), it was found relevant to validate T cell epitope/HLA complexes as actionable targets to ensure efficacy of the above mentioned therapies. Preferably, it may not be sufficient to use HLA binding prediction algorithms to define actionable targets, as these are not precise enough and miss many binding peptides (cf. Table 2). Thus, not only HLA binding of each stimulation peptide referred to herein was experimentally validated, but, furthermore, stimulation peptides were assessed with PBMCs from healthy human donors for T cell memory responses. The thus defined immunogenic peptides must have been naturally processed and presented in a prior HPV16 infection. Also, it is preferred to validate the HLA presentation of putative stimulation peptides directly on the tumor (or other HPV-positive) cells. This can be achieved by a mass-spectrometry-based technology, immunopeptidomics. Thus, in particular the aforesaid two methods in combination can identify good candidate peptides for eliciting a cellular immune response against HPV16-related virus-infected cells and inappropriate cellular proliferations caused by them.

The definitions made above apply mutatis mutandis to the following. Additional definitions and explanations made further below also apply for all embodiments described in this specification mutatis mutandis.

The present invention also relates to a method for determining the nucleic acid sequence of at least a part of an immunoreactive agent specifically binding to a stimulation complex, preferably to human host cells infected with an HPV16-related virus, said method comprising stimulating, identifying, and optionally enriching, immune cells specifically binding to a stimulation complex, preferably to HPV16-related virus-infected host cells, according to a method as specified herein above and the further step of sequencing at least a part of at least one polynucleotide encoding said immunoreactive agent in said immune cell.

The method for determining the nucleic acid sequence of the present invention is an in vitro method. Moreover, it may comprise steps in addition to those explicitly mentioned above. In particular, the sequence information obtained may be used in the construction of an expression construct expressing at least one chain of an immunoreactive reagent recognizing said stimulation peptide. Moreover, one or more of said steps may be aided or performed by automated equipment.

The term “determining the sequence of at least a part of an immunoreactive reagent” is understood by the skilled person. Sequencing may be accomplished by any sequencing method deemed appropriate by the skilled person. Preferably, said sequencing uses mRNA or cDNA encoding at least one polypeptide comprised in the immunoreactive reagent as a template. As the skilled person knows, the immunoreactive reagent may be comprised of one or more than one polypeptide, preferably as specified herein above, so it may be necessary to determine the sequence of more than one polynucleotide encoding an immunoreactive reagent. Preferably, the parts of the polynucleotides encoding said immunoreactive reagent, preferably the T cell receptor, sequenced comprise at least CDR3, preferably at least the CDRs; thus, preferably, at least CDR1 to 3 of each chain of the immunoreactive reagent are sequenced. It is, however, also envisaged that the variable domains of the immunoreactive reagent are sequenced, or that two or more complete chains of the immunoreactive agent are sequenced.

The term “immunoreactive agent”, as used herein, relates to each and every molecule recognizing, i.e. preferably specifically binding to, a stimulation complex, preferably to a human host cell infected with an HPV16-related virus. Thus, the immunoreactive reagent may e.g. be a T cell receptor or an immunoglobulin.

As used herein, the term “T cell receptor” relates to the receptor present on and mediating recognition of antigens by T cells, consisting of an alpha chain and a beta chain or a gamma and a delta chain, preferably of an alpha and a beta chain. The structure of the T cell receptor is known to the skilled person; preferably, each chain of the T cell receptor comprises a cytoplasmic domain, a transmembrane domain, a constant domain, and a variable domain, wherein the variable domain confers specificity to an antigen. As used herein, the cytoplasmic domain if present, the transmembrane domain, and the constant domain together may also be referred to as the “non-variable domain” of a T cell receptor chain. Within the variable domain, antigen recognition is essentially determined by complementarity determining regions (CDRs), referred to as CDR1, CDR2, and CDR3. As is known to the skilled person, in contrast to e.g. immunoglobulins, in T cell receptors only CDR3s of the two TCR chains, i.e. e.g. CDR3 of the alpha chain and CDR3 of the beta chain, contact the epitope presented by a MHC, while the two other CDRs essentially only contact the MHC. Thus, in order to obtain information on the structural basis for antigen specificity of a T cell receptor, it preferably is sufficient to obtain sequence information on CDR3 of each of the two chains of the T cell receptor. As the skilled person understands, the T cell receptor on T cells typically is part of a polypeptide complex comprising further polypeptides, which is referred to as “T cell receptor complex”. The T cell receptor complex preferably comprises at least one of CD3delta, CD3gamma, CD3epsilon, and CD3zeta in addition to the T cell receptor. Preferably, the T cell receptor complex is associated with CD8 as a co-receptor. Numbering of amino acids and identification of substructures of TCRs preferably follows the suggestions of the international ImMunoGeneTics (IMGT) database (Lefranc et al. (2003), Dev. Comp. Immunol., 27:55-77), which identifies conserved amino acids essentially always having the same position in a TCR sequence.

As used herein, the term “immunoglobulin”, which may also be referred to as “antibody”, relates to any cell-bound or soluble immunoglobulin from any of the classes IgA, IgD, IgE, IgG, or IgM, or fragments thereof, recognizing the stimulation complex as specified herein above. Antibodies against a predefined antigen can be prepared by well-known methods. Preferably, the immunoglobulin comprises six complementary determining regions. The complementary determining regions of an antibody preferably are different from those of a TCR and preferably are regions in the variable domains of the heavy and light chain of an antibody that define the binding affinity and specificity of the antibody. There are three CDRs for the heavy chain, CDR1-H, CDR2-H and CDR3-H, and three CDRs for the light chain, CDR1-L, CDR2-L, and CDR3-L. Preferably, determining the nucleic acid sequence of at least a part of an immunoglobulin comprises determining at least the sequences encoding said CDRs, preferably all six CDRs.

As the skilled person understands, the sequence information on CDRs of an antibody, e.g. a monoclonal antibody, may be used to provide a primatized, chimerized, or humanized antibody, or a fragment thereof. Also, a single chain antibody, a single-domain antibody, a nanobody, or an antibody fragment, such as Fab, scFab, and the like may be provided Also comprised as antibodies of the present invention are a bi- or trispecific antibody, a synthetic antibody, or a chemically modified derivative of any of the aforesaid antibodies. Also, the aforesaid information may be used to provide CAR T cells carrying sequences recognizing a stimulation complex, e.g. as a single-chain antibodies.

The present invention also relates to a method for producing an immunoreactive agent against a human host cell infected with an HPV16-related virus, said method comprising
(i) stimulating immune cells specifically binding to HPV16-related virus-infected human host cells according to a method as specified herein above;
(ii) identifying and optionally enriching the stimulated immune cells of step (i), preferably according to a method as specified herein above;
(iii) determining the nucleic acid sequence of at least a part of an immunoreactive agent expressed the said immune cells identified and optionally enriched in step (ii), and
(iv) expressing an at least partial nucleic acid sequence encoding the immunoreactive agent, preferably in a an expression system, thereby producing the immunoreactive agent.

Thus, the present invention also relates to a method for producing an immunoreactive agent against a human host cell infected with a HPV16-related virus, said method comprising expressing an at least partial nucleic acid sequence encoding an immunoreactive agent obtained from an immune cell stimulated by a complex comprising a human leukocyte antigen (HLA) and a stimulation peptide (stimulation complex), wherein said stimulation peptide consists of an amino acid sequence selected from SEQ ID NOs:1 to 111, in a preferred embodiment from SEQ ID NOs:1 to 129.

Thus, the present invention also relates to an in vitro method for producing an immunoreactive agent against human cancer cells infected with HPV16-related virus, said method comprising expressing an at least partial nucleic acid sequence encoding an immunoreactive agent obtained from an immune cell stimulated by a complex comprising a human leukocyte antigen (HLA) and a stimulation peptide (stimulation complex), wherein said stimulation peptide
(i) consists of an amino acid sequence selected from SEQ ID NOs:1 and 2 and said HLA is from HLA supertype HLA-A01;
(ii) consists of an amino acid sequence of SEQ ID NO: 11 and said HLA is from HLA supertype HLA-A02,
(iii) consists of an amino acid sequence selected from SEQ ID NOs:34 to 37 and said HLA is from HLA supertype HLA-A03/A11;
(iv) consists of an amino acid sequence selected from SEQ ID NOs:60 and 61 and said HLA is from HLA supertype HLA-A24;
(v) consists of an amino acid sequence selected from SEQ ID NOs:61, 76, and 77 and said HLA is from HLA supertype HLA-B07; or
(vi) consists of an amino acid sequence selected from SEQ ID NOs:61 and 85 to 92 and said HLA is from HLA supertype HLA-B15;
and wherein an HPV16-derived peptide consisting of the same amino acid sequence as said stimulation peptide has been verified to be presented by human HPV16-positive cancer cells.

The term “expressing” in the context of expressing a nucleic acid sequence, is understood by the skilled person. Thus, expressing an at least partial nucleic acid sequence encoding an immunoreactive agent preferably relates to causing expression of said nucleic acid sequence, e.g. in an appropriate expression cell. The term “expression cell”, as used herein, relates to any cell capable of expressing an at least partial nucleic acid sequence encoding an immunoreactive agent, e.g. encoded by an expression construct or vector as specified herein above. Thus, the expression cell preferably is a bacterial cell, such as an E. coli cell, a cell of a unicellular eukaryote, e.g. a yeast cell, a cell of a fungus r an insect cell, or is a mammalian cell, preferably cultured in vitro. Also preferably, expressing is in vitro expressing in an in vitro translation system. More preferably, expressing is expressing in a cell in vivo.

The present invention also relates to an immune cell expressing an immunoreactive agent specifically binding to HPV16-related virus-infected host cells, wherein said immune cell is obtained or obtainable by the method according to a method as specified herein above.

The present invention also relates to an immunoreactive agent against a human cancer cell infected with a HPV16-related virus, said immunoreactive agent specifically binding to a complex comprising a human leukocyte antigen (HLA) and a stimulation peptide (stimulation complex), wherein said stimulation peptide consists of an amino acid sequence selected from SEQ ID NOs:1 to 111, wherein said immunoreactive reagent is preferably produced or producible according to a method as specified elsewhere herein. Preferably, the immunoreactive agent specifically binds to an immune complex as specified herein below in any one of Tables 1(i) to 1(vi), preferably a complex selected from those designated as Complex ID NOs: 1, 2, 34-38, 61, 62, 78-80, and 89-97, in a preferred embodiment from those designated as Complex ID NOs: 1, 2, 34-38, 61, 62, 77-80, 89-97, 132, and 136.

The present invention also relates to a use, preferably in vitro use, of a stimulation peptide consisting of an amino acid sequence selected from SEQ ID NOs:1, 2, 11, 34-37, 60, 61, 76, 77, and 85-92, in a preferred embodiment SEQ ID NOs:1, 2, 11, 34-37, 60, 61, 75-77, 85-92, 121, and 136, verified to be presented by human HPV16-positive cancer cells, and verified to stimulate human anti-stimulation peptide immune cells when presented as a human leukocyte antigen (HLA)-complex, for stimulating, identifying, and optionally enriching, immune cells specifically binding to HPV16-related virus-infected human host cells.

The present invention further relates to an immune cell and/or immunoreactive agent as specified herein above for use in medicine, preferably for use in treating HPV16-related virus-related inappropriate cellular proliferation.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., as described herein below. Preferably, treating cancer is reducing tumor burden in a subject. As will be understood by the skilled person, effectiveness of treatment of e.g. cancer is dependent on a variety of factors including, e.g. cancer stage and cancer type. Preferably, treating causes inappropriately proliferating cells, preferably neoplastic cells, more preferably cancer cells, to be recognized by T-cells of the subject. Thus, preferably, treating has the effect of killing tumor cells, causing a tumor to stop growing, in particular causing tumor cells to stop proliferating, more preferably causing regression of a tumor, more preferably causing a tumor to resolve. As used herein, the above relates to treating other HPV16 related virus-positive inappropriate cellular proliferations mutatis mutandis.

Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%.

The p-values are, preferably, 0.1, 0.05, 0.01, 0.005, or 0.0001. Preferably, the treatment shall be effective for at least 60%, at least 70%, at least 80%, or at least 90% of the subjects of a given cohort or population.

In view of the above, the following embodiments are particularly envisaged:

Embodiment 1: A method for stimulating immune cells specifically binding to human host cells infected with an HPV16-related virus, said method comprising
(A) contacting a complex comprising a human leukocyte antigen (HLA) and a stimulation peptide (stimulation complex) with immune cells, and
(B) thereby stimulating immune cells specifically binding to HPV16-related virus-infected host cells,
wherein said stimulation peptide consists of an amino acid sequence selected from SEQ ID NOs:1 to 111, in a preferred embodiment SEQ ID NOs:1 to 129.

Embodiment 2: The method of embodiment 1, wherein said stimulation peptide has been verified to stimulate human anti-stimulation peptide immune cells when presented as a human leukocyte antigen (HLA)-complex.

Embodiment 3: The method of embodiment 1 or 2, wherein said human host cells are human cancer cells infected with an HPV16-related virus, wherein an HPV16-derived peptide consisting of the same amino acid sequence as the stimulation peptide has been verified to be presented by human HPV16-positive cancer cells, and wherein said stimulation peptide consists of an amino acid sequence selected from SEQ ID NOs:1, 2, 11, 34-37, 60, 61, 76, 77, and 85-92.

Embodiment 4: The method of any one of embodiments 1 to 4, wherein said stimulating is activating said immune cells and/or increasing the number of immune cells specifically binding to said stimulation complex.

Embodiment 5: The method of any one of embodiments 1 to 4, wherein said stimulation complex is comprised on an antigen presenting cell (APC) or an artificial APC presenting said at least one stimulation complex or is an HLA oligomer comprising said stimulation peptide.

Embodiment 6: The method of any one of embodiments 1 to 5, wherein said method is a method for stimulating and identifying immune cells specifically binding to a stimulation complex, and wherein said method further comprises (C) identifying stimulated immune cells stimulated in step (B).

Embodiment 7: The method of any one of embodiments 1 to 6, wherein said method is a method for stimulating and enriching immune cells specifically binding to human host cells infected with an HPV16-related virus and wherein said method further comprises step (D) enriching stimulated immune cells stimulated in step (B) and optionally identified in step (C).

Embodiment 8: The method of embodiment 6 or 7, wherein said method further comprises a step of contacting the immune cells of step (B) with a derivative of said stimulation complex carrying a detectable label and identifying and optionally enriching immune cells binding to said stimulation complex by said detectable label.

Embodiment 9: The method of any one of embodiments 1 to 8, wherein said method is an in vitro method.

Embodiment 10: The method of any one of embodiments 1 to 8, wherein step (A) comprises administering said stimulation complex to a subject.

Embodiment 11: The method of embodiment 10, wherein said subject is a non-human animal and wherein said subject preferably is sacrificed after step (C) or (D).

Embodiment 12: A method for determining the nucleic acid sequence of at least a part of an immunoreactive agent specifically binding to human host cells infected with an HPV16-related virus, said method comprising stimulating, identifying, and optionally enriching, immune cells specifically binding to HPV16-related virus-infected host cells according to the method of any one of embodiments 1 to 11 and the further step of sequencing at least a part of at least one polynucleotide encoding said immunoreactive agent in said immune cell.

Embodiment 13: A method for producing an immunoreactive agent against a human host cell infected with an HPV16-related virus, said method comprising
(i) stimulating immune cells specifically binding to HPV16-related virus-infected human host cells according to the method of any one of embodiments 1 to 5 or 9 to 11;
(ii) identifying and optionally enriching the stimulated immune cells of step (i), preferably according to the method of any one of embodiments 6 to 10;
(iii) determining the nucleic acid sequence of at least a part of an immunoreactive agent expressed the said immune cells identified and optionally enriched in step (ii), and
(iv) expressing an at least partial nucleic acid sequence encoding the immunoreactive agent, preferably in a an expression system, thereby producing the immunoreactive agent.

Embodiment 14: A method for producing an immunoreactive agent against a human host cell infected with a HPV16-related virus, said method comprising expressing an at least partial nucleic acid sequence encoding an immunoreactive agent obtained from an immune cell stimulated by a complex comprising a human leukocyte antigen (HLA) and a stimulation peptide (stimulation complex), wherein said stimulation peptide consists of an amino acid sequence selected from SEQ ID NOs:1 to 111.

Embodiment 15: An in vitro method for producing an immunoreactive agent against human cancer cells infected with HPV16-related virus, said method comprising expressing an at least partial nucleic acid sequence encoding an immunoreactive agent obtained from an immune cell stimulated by a complex comprising a human leukocyte antigen (HLA) and a stimulation peptide (stimulation complex), wherein said stimulation peptide
(i) consists of an amino acid sequence selected from SEQ ID NOs:1 and 2 and said HLA is from HLA supertype HLA-A01;
(ii) consists of an amino acid sequence of SEQ ID NO: 11 and said HLA is from HLA supertype HLA-A02,
(iii) consists of an amino acid sequence selected from SEQ ID NOs:34 to 37 and said HLA is from HLA supertype HLA-A03/A11;
(iv) consists of an amino acid sequence selected from SEQ ID NOs:60 and 61 and said HLA is from HLA supertype HLA-A24;
(v) consists of an amino acid sequence selected from SEQ ID NOs:61, 76, and 77 and said HLA is from HLA supertype HLA-B07; or
(vi) consists of an amino acid sequence selected from SEQ ID NOs:61 and 85 to 92 and said HLA is from HLA supertype HLA-B15;
and wherein an HPV16-derived peptide consisting of the same amino acid sequence as said stimulation peptide has been verified to be presented by human HPV16-positive cancer cells.

Embodiment 16: The method of embodiment 14 or 15, herein said method has a further feature of at least one of embodiments 1 to 13.

Embodiment 17: The method of any one of embodiments 1 to 16, wherein said immune cells are T cells expressing a T cell receptor specifically binding to the immune complex and/or are B cells specifically binding to the immune complex.

Embodiment 18: The method of any one of embodiments 1 to 17, wherein said immune cells are T cells, preferably CD8+ T cells, more preferably cytotoxic CD8+ T cells.

Embodiment 19: The method of any one of embodiments 1 to 16, wherein said HPV16-related virus is HPV16, HPV31, HPV33, HPV35, HPV52, HPV58, or HPV67, more preferably is HPV16.

Embodiment 20: An immune cell expressing an immunoreactive agent specifically binding to HPV16-related virus-infected host cells, wherein said immune cell is obtained or obtainable by the method according to the method of any one of embodiments 1 to 11.

Embodiment 21: The immune cell of embodiment 13, wherein said immune cell is a T cell and said immunoreactive agent is a T cell receptor or wherein said immune cell is a B cell an said immunoreactive agent is an immunoglobulin.

Embodiment 22: An immunoreactive agent against a human cancer cell infected with a HPV16-related virus, said immunoreactive agent specifically binding to a complex comprising a human leukocyte antigen (HLA) and a stimulation peptide (stimulation complex), wherein said stimulation peptide consists of an amino acid sequence selected from SEQ ID NOs:1 to 111.

Embodiment 23: The immunoreactive agent of embodiment 22, produced or producible according to the method of any one of embodiments 13 to 19.

Embodiment 24: Use, preferably in vitro use, of a stimulation peptide consisting of an amino acid sequence selected from SEQ ID NOs:1, 2, 11, 34-37, 60, 61, 76, 77, and 85-92, verified to be presented by human HPV16-positive cancer cells, and verified to stimulate human anti-stimulation peptide immune cells when presented as a human leukocyte antigen (HLA)-complex, for stimulating, identifying, and optionally enriching, immune cells specifically binding to HPV16-related virus-infected human host cells.

Embodiment 25: The subject matter of any of the preceding embodiments, wherein said immunoreactive agent is a TCR, an anti-immune complex antibody or a fragment or derivative thereof.

Embodiment 26: The subject matter of any of the preceding embodiments, wherein said immunoreactive reagent is comprised in a T cell.

Embodiment 27: An immune cell and/or immunoreactive agent according to any one of embodiments 20 to 23 for use in medicine, preferably for use in treating HPV16-related virus-related inappropriate cellular proliferation.

All references cited in this specification are herewith incorporated by reference with respect to their entire disclosure content and the disclosure content specifically mentioned in this specification.

The following Examples shall merely illustrate the invention. They shall not be construed, whatsoever, to limit the scope of the invention.

### EXAMPLE 1: METHODS FOR T CELL REACTIVITY ASSESSMENT

### Interferon-γ ELISpot Assay

Prior to the ELISpot assay, ELISpot plates (Millipore Multiscreen-HA membrane sterile plate) were coated with 100 μl of 2 μg/mL anti-human IFN-γ (1-D1K) antibody in sterile PBS and incubated at 4° C. overnight or up to 3 days.

Before setting up the ELISpot assay, the coating antibody was discarded, wells were washed three times with 200 μL sterile PBS and blocked with 200 μL ELISpot medium at standard culture conditions for 1-1.5 h. After blocking, ELISpot medium was discarded and wells were filled with 100 μL antigen solution.

Each (short-term) T cell line (see below) was stimulated in a total of 8 wells: four wells with respective antigen (10 μg/mL single peptides, 0.1% DMSO or CEF peptide pool (1 μg/mL of each peptide)), two wells with concanavalin A (2 μg/ml) as unspecific mitogen-stimulated positive control, and two wells with 0.1% (v/v) DMSO as background control. Short-term antigen-specific T cell lines (described in 1.4) were added in a concentration of 1-2×10⁵ cells in 100 μL per well (total volume of 200 μL/well). The cell numbers of three representative wells (CEF-, DMSO-, peptide-stimulated) were assessed, to enable the calculation of spot forming units (SFU) per 1×10⁶ cells later on. The ELISpot plates were incubated at standard culture conditions for 20-24 h. Moving the plates or the incubator was strictly avoided during incubation to prevent blurred spot formation.

The remaining cell suspension was discarded and ELISpot plates were washed twice with PBS and twice with ELISpot washing buffer. All washes were performed with 200 μL/well. Then, 100 μL/well of 1 ng/mL sterile anti-human IFNγ biotin (7 B6-1) antibody in sterile PBS were added. After two hours of incubation in the dark at room temperature, the antibody was discarded and the plates were washed four times with ELISpot washing buffer. Sterile streptavidin-alkaline phosphatase was diluted by a factor of 1:2000 in sterile PBS and 100 μL/well of this dilution were distributed.

After 1.5 hours of incubation in the dark at room temperature, the antibody was discarded and the plates were washed four times with ELISpot washing buffer. Then, the BCIP/NBT-Plus substrate was filtered through a 0.22 μm filter and 100 μL/well were distributed. The plates were covered with aluminum foil and left at room temperature for 18-20 min until spots developed in the positive control wells. The reaction was stopped by washing the ELISpot plates with tap water and the plates were allowed to air dry for a minimum of 1.5 h.

### Analysis of ELISpot Plates

In order to obtain objective results about the number of SFU per well, a CTL automated ELISpot plate reader was used to count the ELISpot plates. The SmartCount™ counting mode of the ImmunoSpot 5.1.36 Professional DC software was used for the analysis. The settings were adjusted for each donor individually to account for donor specific variance. In general, the background balance was set to values between 0 and 10 and the spot separation was set to values between 1 and 4. The automatically determined minimum spot size was adjusted to exclude small background spots based on negative control wells. Similarly, maximum spot size was automatically determined based on positive control wells. A quality control for each well was done manually to detect and correct errors of the counting algorithm caused by uneven development or fibers. Based on the counting results, the mean SFU per 1×10⁶ cells and the stimulation index (SI), defined as the fold change of the mean SFU of peptide-stimulated relative to DMSO background wells, were calculated for each peptide-specific cell line. Responses with SI>2 and SFU per 1×10⁶ cells >100 as well as responses with SI>4 and SFU>50 were considered positive.

### T Cell Phenotyping and Intracellular Cytokine Staining

To characterize and quantify the responsive cells upon peptide stimulation, the peptide-specific T cell cultures with positive ELISpot results were further analyzed with T cell phenotyping and intracellular cytokine staining (ICS) assessed by flow cytometry.

The cells of the respective continued short-term T cell culture as well as the DMSO-stimulated and an unresponsive T cell line were harvested into 2 mL reaction tubes and centrifuged at 400 xg for 10 min. The supernatant was discarded and the pellet of the unresponsive T cell line was resuspended in 100 μL ELISpot medium and used as unstimulated and unstained control. The pellets of the responsive and the DMSO-stimulated T cell lines were resuspended in 200 μL ELISpot medium and split equally into two wells of a V-bottom 96-well plate. One well of the responsive T cell lines was re-stimulated with 10 μg/mL of its specific peptide while the other well was mock stimulated with 0.1% (v/v) DMSO. As positive control, one well of the DMSO-stimulated T cell line was activated by treatment with 20 ng/ml Phorbol 12-myristate 13-acetate and 1 μM Ionomycin. Then, 1:10 diluted GolgiStop (containing monensin) and 1:15 diluted GolgiPlug (containing brefeldin A) were added to all wells to inhibit intracellular transport processes. Monensin inhibits the trans-Golgi transport, while brefeldin A inhibits transport between the ER and the Golgi (Chardin and McCormick, 1999; Mollenhauer et al., 1990). The stimulated cells were incubated for 5 h at standard culture conditions.

After incubation, the cells were resuspended, centrifuged at 400 xg for 5 min and the supernatant was discarded. Then, the cells were resuspended in 50 μL cold staining buffer (unstained) or 50 μL cold staining buffer containing the phenotyping antibodies against CD3, CD4, CD8 and the LIVE/DEAD™ Fixable Near-IR Dead Cell Stain (unstimulated and stimulated) and incubated at 4° C. for 30 min. After the surface staining, the cells were washed twice with 200 μL staining buffer and centrifugation at 1400 rpm, followed by fixation with 1% paraformaldehyde (PFA) solution for 15 min at 4° C. All following centrifugation steps were carried out at 1400 rpm for 5 min. After fixation, the cells were again washed twice with 200 μL cold staining buffer and were then resuspended in 100 μL 1× perm/wash buffer diluted in PBS (provided is 10×) for 15 min at 4° C. for cell membrane permeabilization. Subsequently, the plate was centrifuged and the supernatant was discarded. The cells were resuspended in 50 μL perm/wash buffer diluted in PBS (unstained) or 50 μL perm/wash buffer containing the intracellular cytokine antibodies against IFNγ, TNFα and granzyme B (unstimulated and stimulated) for 30 min at 4° C. After intracellular cytokine staining, the cells were washed twice with 200 μL 1× perm/wash buffer. Then 100 μL fix/perm solution (provided in the BD Cytofix/Cytoperm kit) were added per well. After 20 min of incubation at 4° C., the cells of each well were washed twice with 200 μL 1× perm/wash buffer before being resuspended in 100 μL staining buffer and stored at 4° C. overnight.

OneComp eBeads™ (eBeads) and ArC™ Amine Reactive Compensation beads (ArC beads) were stained as compensation controls. For each antibody, 50 μL of the eBeads suspension were stained with the same dilution as used for staining the cells. The ArC beads were stained with 1 μL LIVE/DEAD™ Fixable Near-IR Dead Cell Stain stock solution in 50 μL staining buffer. The beads were incubated at 4° C. for 30 min and were then washed two times with 1 mL staining buffer and centrifugation at 400×g. Finally, the stained beads were resuspended in 400 μL staining buffer, one drop of ArC™ negative beads was added to the labelled ArC beads, and all beads were stored at 4° C. overnight.

On the next day, the samples and the single stained compensation controls were acquired at a BD FACS Canto™ II analyzer using the BD FACS Diva Software Version 6. The obtained data were analyzed with the FlowJo software Version 10 by applying the same gating strategy to all samples.

### Short-Term T Cell Lines

Short-term antigen-specific T cell lines were generated from PBMCs in order to expand antigen-specific memory T cells induced by previous natural HPV16 infections. To do so, T cells were stimulated with HLA-matched HPV16 E6- or E7-derived HLA-ligands. To set up short-term T cell lines, human PBMCs were thawed and subsequently resuspended in T cell medium supplemented with recombinant human interleukin-7 (rhIL-7) (10 ng/mL) and recombinant human interleukin-15 (rhIL-15) (20 ng/mL). The cells were counted and 1-2×10⁶ cells per antigen stimulation were seeded in wells of 24-well plates in a total volume of 2 mL per T cell line. To obtain antigen-specific T cell lines, 10 μg/mL of HLA-binding HPV16 E6/E7-derived peptides were added to each well. As peptide-specific positive control, one cell line was stimulated with a peptide pool consisting of 23 well described HLA class I-restricted epitopes of the widespread viruses human cytomegalovirus (CMV), Epstein-Barr-Virus (EBV) and Influenza A (CEF peptide pool) (Currier et al., 2002) at a concentration of 1 μg/mL for each peptide. As single peptide controls, two cell lines were stimulated with 10 μg/mL HLA-matched EBV- and CMV-derived peptide. As peptide-specific negative control, a cell line was stimulated with 10 μg/mL HLA-matched human immunodeficiency virus (HIV)-derived peptide (10 μg/mL), while treatment with 0.1% (v/v) dimethyl sulfoxide (DMSO, peptide solvent) served as unspecific negative control. The stimulated PBMCs were cultivated at standard culture conditions.

On day 3, the cells were fed with recombinant human interleukin-2 (rhIL-2) (20 U/mL) and rhIL-15 (20 ng/mL) to promote proliferation of T cells. Seven days after culture setup, a half-medium change was performed. For this, the cells were pelleted on the well bottom by centrifugation at 300 xg for 5 min and 1 mL of the supernatant was carefully removed from each well. Then, 1 ml fresh T cell medium supplemented with rhIL-2 (20 U/mL final) and rhIL-15 (20 ng/mL final) was added and cells were resuspended.

After 12 days of culturing, half of the cells of each culture was used to set up ELISpot assays as described in 1.1. The remaining cells were fed with 1 mL ELISpot medium and cultivated for two more days until the cells were used for T cell phenotyping and intracellular cytokine staining (ICS) as described in 1.2.

### Cytotoxicity Assay

The flow cytometry based VITAL FR cytotoxicity assay was used as published by Stanke et al. (Stanke et al., 2010) to assess whether peptide-specific T cells are able to specifically kill HPV-transformed target cells. Briefly, two HLA-A2⁺ cell lines, a HPV16-negative (HPV16⁻) control cell line (C33A) and a HPV16-positive (HPV16⁺) target cell line (CaSki) were fluorescently labeled with either FarRed or CFSE and were co-incubated with peptide-specific CD8⁺ T cells as effector cells. After 48 h co-incubation, the fluorescently labeled cells were analyzed by flow cytometry to determine the surviving cells and calculate the specific cytolytic function of the effector cells by the ratio of target and control cells.

CaSki and C33A cells were fluorescently labeled with CFSE and FarRed, respectively. Cells of both cell lines were harvested and resuspended in plain RPMI medium at a concentration of 1×10⁶ cells/mL. For cell labeling, 5 μM CSFE were added to CaSki cells and 0.25 μM FarRed were added to C33A cells. The cells were incubated at 37° C. and the reaction was stopped after 10 min by addition of 50 mL RPMI with 10% FBS. The cells were centrifuged at 300 xg for 5 min, the supernatant was discarded and the cells were resuspended in their cell culture medium. The labeled cells were seeded in T25 cell culture flasks with 1-2×10⁶ cells/flask.

On the next day, the labeled CaSki and C33A as well as the epitope-specific semi-long-term T cell lines (described in 1.6) were harvested. CD8⁺ T cells were isolated from the epitope-specific semi-long-term T cell lines using the MACS CD8⁺ T cell isolation kit (Miltenyi Biotec) according to the manufacturer's protocol for LS columns. The CD8⁺ T cells were resuspended in T cell medium and added to wells of a F-bottom 96-well plate in serial dilutions to achieve triplicates of 6×10⁴, 3×10⁴, 1.5×10⁴, 0.75×10⁴ and 0.375×10⁴ cells per well. The CaSki and C33A cells were mixed 1:1 in T cell medium and 3×10³ cells were added to each well containing T cells. Thereby effector:target (E:T) ratios of 1:20, 1:10, 1:5, 1:2.5 and 1:1.25 were achieved. Additionally, triplicates of mixed target cells without T cells were seeded, which is referred to as E:T 0. After 48 h incubation at 37° C., 5% CO₂, the cells were harvested and analyzed at a BD FACS Canto™ II analyzer using the BD FACS Diva Software Version 6. The obtained data were analyzed with the FlowJo software Version 10 by applying the same gating strategy to all samples. Subsequently, the equations (1) and (2) given below were applied in Excel (Microsoft Office 2016) to calculate specific killing.

$$ratio of target cells ( \% ) = \frac{frequency of CaSki}{frequency of C 33 A} \qquad ( 1 )$$

$$specific killing ( \% ) = 100 - ( \frac{ratio of target cells in presence of T cells}{ratio of target cells without T cells} * 100 ) \qquad ( 2 )$$

### Semi Long-Term T Cell Lines

Semi long-term T cell lines were set up to obtain high numbers of T cells, which are required for the analysis of epitope specific cytotoxic T cell responses in VITAL FR cytotoxicity assays. Autologous DCs were generated from PBMCs as described in 1.7 and loaded with the desired HPV16 peptide. PBMCs of the same donor were thawed and resuspended in T cell medium supplemented with rhIL-7 (10 ng/mL) and rhIL-15 (20 ng/mL). 1×10⁷ PBMCs/well were seeded to a 24-well plate in a total volume of 1 mL/well. Peptide-loaded DCs were added in a ratio of 50:1 (PBMCs:DCs) in 1 mL rhIL-7 (10 ng/mL) and rhIL-15 (20 ng/mL) supplemented T cell medium to achieve a final volume of 2 mL. The cell cultures were fed with rhIL-2 (final concentration: 40 U/mL) every other day. If the medium turned yellow the feeding was combined with a half medium change with 1 mL fresh IL-2 supplemented T cell medium. After eight days, the peptide stimulation with peptide-loaded DCs was repeated and cultivation was continued. On day 15, the T cell lines were harvested and used for CD8⁺ T cell isolation and cytotoxicity assays.

### Generation of Autologous DCs

To generate autologous DCs, frozen human PBMCs were thawed. After thawing, cells were counted and resuspended in DC medium to reach a density of 5×10⁶ cells/mL. From this suspension, 1×10⁷ cells in 2 mL DC medium were seeded per well in a 6-well plate.

After 3 hours incubation at 37° C., 5% CO₂ the supernatant was removed and transferred to a 50 mL tube. The wells were gently washed with 1 mL warm DC medium to remove non-adherent cells and the washing medium was collected together with the supernatant. The remaining adherent cells were fed with 2 mL fresh DC medium supplemented with 100 ng/mL granulocyte-macrophage colony-stimulating factor (GM-CSF) and 50 ng/mL interleukin-4 (IL-4) for further cultivation. The collected non-adherent cells containing T and B cells were counted and frozen for future use.

After three days of culturing, the cells were fed with 0.5 mL fresh DC medium supplemented with 100 ng/mL GM-CSF and 50 ng/mL IL-4. Six days after the cultures were started, the DCs were maturated by adding 1000 U/ml tumor necrosis factor α (TNFα), 10 ng/ml interleukin-1β (IL-1β), 10 ng/ml interleukin-6 (IL-6), 1 μM Prostaglandin E2 (PGE2) and 1 μg/ml lipopolysaccharide (LPS) to each well. The cells were incubated with this maturation cocktail for 48 hours and harvested by gentle scraping with a cell scraper.

### Live-Cell Imaging-Based Cytotoxicity Assay

In addition to the VITAL FR assay described herein above, a newly established live-cell imaging-based cytotoxicity assay was applied. In this assay, transiently red labeled HPV16 transformed target cells were co-incubated with either HPV16-peptide-specific or non-specific CD8+ T cells. Apoptotic cells were stained with a green caspase dye. By live cell imaging, the frequency of apoptotic target cell (stained in red and green) was analyzed over time. Peptide-specific killing was detected by increased frequencies of apoptotic target cells in co-culture with HPV16-peptide-specific CD8+ T cells compared to co-culture with non-specific CD8+ T cells. This highly sensitive assay was used to detect killing mediated by low frequent CTL populations and, in contrast to the common endpoint assays, it enables the time course analysis of the CTL mediated cytotoxicity. Moreover, it is very flexible and can easily be adapted for other target cell lines of interest.

### EXAMPLE 2: IMMUNOPRECIPITATION (IP) AND LC/MS ANALYSIS OF PEPTIDES PRESENTED BY CULTURED CELLS

### Immunoprecipitation of HLA-Presented Peptides

HLA immunoprecipitation (IP) was performed according to previously published protocols (Bassani-Sternberg et al., 2016; Chong et al., 2018). Briefly, HPV16+ cells were lysed with a lysis buffer containing 1% N-octyl-β-D glucopyranoside, 0.25% Na-Deoxycholate, protease inhibitor cocktail (Sigma-Aldrich, Mannheim, Germany)/PMSF (Carl Roth, Karlsruhe, Germany) in PBS. After centrifugation at 40,000 xg, 4° C., for 30 min, HLA-peptide complexes were immuno-precipitated by incubation with mouse anti-human HLA-A,B,C monoclonal antibody (clone W6/32, Biolegend, San Diego, CA, USA) or in some cases HLA-type-specific antibody (HLA-A2: clone BB7.2, HLA-A3: clone GAP A3, HLA-A11/A24: clone Hb 164, HLA-B7: clone BB7.1) crosslinked to protein G Sepharose beads (Cytiva, Marlborough, MA, USA) or Protein A—Sepharose™ 4B (Invitrogen Corporation, Camarillo, CA, USA) for 4 h at 4° C. under constant mixing on a rotating wheel. Supernatant was discarded after centrifugation at 3200 xg, for 3 min at RT. Pelleted HLA-peptide complexes bound to antibody-beads were washed 3 times in each of the following steps: first with ice-cold 20 mM Tris-HCl pH 8 containing 150 mM NaCl followed by the same buffer but with 400 mM NaCl and finally with 20 mM Tris-HCl alone. Peptides were eluted from antibody-beads bound to HLA by 0.3% TFA. Alkylation of cysteine-containing peptides was optionally performed at this stage. 1 ml of IP eluate was reduced with 100 μl HEPES/50 mM TCEP with incubation for 10 min at RT. Alkylation was performed with 100 μl of 400 mM iodoacetamide (IAA) with incubation in the dark for 20 min at RT. Alkylation was quenched with 100 μl of 1 M HEPES/50 mM TCEP with incubation for 3 min at RT. The sample was acidified again with 60 μl of 10% TFA. Resulting peptides were desalted by reverse-phase purification using a SepPak 96-well plate (Waters, Milford, MA, USA). While loaded on the sorbent, an oxidation reaction was performed. Performic acid was prepared by combining 5 μl of 30% H₂O₂ and 45 μl of 10% formic acid in a glass vial and letting it rest for 5 min. Sample oxidation was performed by brief application of 1 ml of the performic acid solution in the well, which was washed out afterwards with 1 ml of 0.1% TFA. Peptides eluted in 28% ACN in 0.1% TFA were dried by vacuum centrifugation (Concentrator plus, Eppendorf, Hamburg, Germany).

### Direct Infusion and LC-MS

The set of predicted target peptides were acquired as stable isotope labelled (SIL) peptides from commercial suppliers (crude synthesis product; isotope purity >99 atom % ¹³C and ¹⁵N). To alkylate cysteine-containing peptides, 2 ng per peptide of vacuum dried peptide mixture was resolubilized in 20 μl 100 mM HEPES with 3 min sonication in a 0.5 μl protein LoBind microcentrifuge tube. 5 μl (500 pmol per peptide) of the sample was reduced by addition of 2 μl HEPES/50 mM TCEP with incubation for 10 min at RT. Alkylation was performed by addition of 2 μl 400 mM IAA with incubation in the dark for 20 min at RT. Alkylation was quenched by addition of 2 μl 100 mM HEPES/50 mM TCEP. The sample was acidified by adding 0.3% TFA to a volume of 500 μl and desalted using a 100 mg sorbent well of a 96-well SepPak plate (Waters, Milford, MA, USA). The elution step was performed with 80% ACN/0.1% TFA.

First, analysis by direct infusion was performed in order to optimize normalized collision energy (NCE) for each single peptide in a mixture. NCE values were ranging from 4% to 42% and each value, in 2% steps, was measured 4 times for four charge states (1+ to 4+) of each peptide. Orbitrap Exploris 480 (Thermo Fisher Scientific) in targeted MS2 scan (PRM) was operated using 30K resolution at 200 m/z, standard automated gain control (AGC) target, 350 ms maximum injection time (IT), 1 micro scan in centroid data acquisition mode. For all measurements, isolation window was 0.4, 0.7, 1 or 1.5 m/z, depending on the mass of the precursor, as recommended by the manufacturer. Ion chromatograms of all expected transitions were extracted with 12 ppm mass tolerance from the centroided spectra and deconvoluted using purpose-made R scripts. The best NCE value was selected so that the intensity of any 5th most intense transitions was maximized, thereby maximizing the chance of detecting at least 5 transitions. The retention time (RT) for each peptide was determined by LC-MS in DDA mode with an inclusion list of target peptides in all four charge-states. The MS resolution was set to 120K, 3e6 AGC target, 50 ms maximum IT and the LC operated as described below.

For LC-MS, samples were dissolved in 2.5 to 5 μl of 5% ACN in 0.1% TFA with 50 fmol of Peptide Retention Time Calibration (PRTC) Mixture (88321, Pierce™) spiked in, followed by 3 min bath sonication. All samples were analysed by liquid chromatography (U-3000, Thermo Fisher Scientific) coupled to Orbitrap Exploris 480 (Thermo Fisher Scientific). 150 fmol of BSA digest (88341, Pierce™)) and 50 fmol PRTC Mixture (free of target peptides) was systematically injected before IP samples as a negative control to test for the absence of any artefact, e.g. due to possible carryover of light contamination from SIL peptides after prior quality control tests (Salek et al. 2022). The LC gradient consisted of multiple segments. First, the solvent B (100% ACN, 0.1% FA) went from 2% to 6% and 94% A (0.1% FA in H₂O) in 5 min and then to 28.5% B in 75 min. It reached 80% in 4 min followed by 5 min wash at 80% B. Finally, the column was equilibrated for 11 min at 2% B.

For LC-MS experiments, the MS was acquired with 60K resolution at 200 m/z, over the mass range from 150 to 1450 m/z, 3e6 AGC target and 25 ms maximum IT. MS2 data was acquired with PRM scans using 60 k or 120 k resolution at 200 m/z. Target precursor list was provided with preselected charge states, the corresponding m/z and optimized collision energy values for each target, their expected retention time (+/−1 to 3 min) pre-defined with SIL peptide. The normalized AGC target was set to 1000% (or 1e6). The maximum injection time mode was set to dynamic, allowing sampling of a minimum of 5 points across the chromatographic peak. The dynamic RT feature using the Pierce PRTC mixture was active.

### Data Analysis

LC-MS data was analysed with the Skyline software v. 20.2 (MacLean et al., 2010). A minimum of top 5 intensity product ions were extracted with 7 ppm mass tolerance. Detected peaks were manually curated. Light peaks were discarded when peak retention times or shapes did not match with the heavy reference, when the normalized spectral contrast angle (NSCA) (Toprak et al., 2014) was low, or when too few transitions were detected.

The findings of the present invention are summarized also in Table 2 below; as shown, there are a vast number of peptides derivable from HPV 16 E6 and E7 (approx. 9000). Prediction algorithms identify approx. ⅓ of the peptides actually binding to HLA, and wrongly predict binders in about 50% of cases. Moreover, of immunogenic peptides only ½ are correctly predicted, and of peptides actually presented on HPV16 positive tumor cells, only approx. ¼ was correctly predicted.

Detection of Cystein-Containing Peptides For the detection of HPV16-derived T-cell epitopes, an immunopeptidomics workflow was established for which each individual candidate epitope is assessed by a scientist and an optimized targeting strategy is devised. Due to HPV16 proteins E6 and E7 being rich in cysteine, 148 of the 242 candidate peptides contained cysteine (cf. Table 3 below). Contrary to the usual proceeding in the art, cf. e.g. Blatnik et al. ((2018, loc.cit.)), cysteine-containing peptides were not excluded from the analysis. This required an adapted strategy as the detection of cysteine-containing peptides is particularly challenging for MS experiments due to the propensity of the thiol side chain to undergo oxidative modifications. The resulting dispersion of cysteine-containing peptides into various forms does not allow for the required sensitive detection in targeted experiments. For this reason, the preceding studies on HPV16 did not target any cysteine-containing peptides and this limitation was overcome by the integration of a cysteine alkylation reaction into the experiment.

TABLE 3
Numbers of cysteine-containing candidate peptides.

| | Candidate peptide count | |
|---|---|---|
| Total | 242 | |
| Not cysteine-containing | 148 | (61%) |
| Cysteine-containing | 94 | (39%) |

TABLE 1
Preferred stimulation peptide/HLA combinations; Protein/Position: HPV16 protein and amino
acid position the peptide is derived from, deviations from the HPV16 reference sequence
are indicated if applicable; presentation: HLA-presentation of the peptide by HPV16-
positive cells shown by MS; ELISPOT: Immungenicity of the peptide shown by IFN-gamma
ELISPOT analysis; cytokine: Immunogenicity of the peptide shown
by intracellular cytokine staining (IFN-gamma, TNF-alpha);
cytotox: Ability of peptide-specific T cells to kill HPV16-positive cells.
* Highlights data from published resources

| | | | | | | | | |
|---|---|---|---|---|---|---|---|---|
| | | SEQ | HLA | Complex | | | | |
| | Peptide | ID | restric- | ID | | | | presenta- |
| Protein/Position | sequence | NO | tion | NO | ELISpot | cytokine | cytotox | tion |

Table 1(i): Peptides presented via HLA-A01

| | | | | | | | |
|---|---|---|---|---|---|---|---|
| E6/80-88 | ISEYRHYCY | 1 | A01 | 1 | +* | | + |
| E7/2-11 | HGDTPTLHEY | 2 | A01 | 2 | +* | | + |
| E6 E36Q/29-39 | TIHDIILQCVY | 3 | A01 | 3 | + | + | |
| E6 A68G/67-77 | YGVCDKCLKFY | 4 | A01 | 4 | + | + | |
| E6 H85Y/82-91 | EYRYYCYSLY | 5 | A01 | 5 | + | + | |
| E7/14-23 | DLQPETTDLY | 6 | A01 | 6 | + | + | |
| E7/19-27 | TTDLYCYEQ | 7 | A01 | 7 | + | + | |
| E6/78-88 | SKISEYRHYCY | 8 | A01 | 8 | + | | |
| E6/81-91 | SEYRHYCYSLY | 9 | A01 | 9 | + | | |
| E7/18-25 | ETTDLYCY | 10 | A01 | 10 | + | | |
| E7/19-28 | TTDLYCYEQL | 112 | A01 | 121 | + | | |
| E6/81-88 | SEYRHYCY | 113 | A01 | 122 | + | | |
| E6 D32E/30-39 | IHEIILECVY | 114 | A01 | 123 | + | | |
| E6 A68G/68-77 | GVCDKCLKFY | 115 | A01 | 124 | + | | |

Table 1(ii): Peptides presented via HLA-A02

| | | | | | | | | |
|---|---|---|---|---|---|---|---|---|
| E7/66-74 | RLCVQSTHV | 11 | A02 | 11 | + | | + | + |
| E7/83-93 | LMGTLGIVCPI | 12 | A02 | 12 | + | + | + | |
| E7/11-18 | YMLDLQPE | 13 | A02 | 13 | + | | + | |
| E7/84-93 | MGTLGIVCPI | 14 | A02 | 14 | + | | + | |
| E6 D32E/25-33 | ELQTTIHEI | 15 | A02 | 15 | + | + | | |
| E6 D32E/28-38 | TTIHEIILECV | 16 | A02 | 16 | + | 1 | | |
| E6 D32E, I34R/28-38 | TTIHEIRLECV | 17 | A02 | 17 | + | + | | |
| E6 D32E/29-38 | TIHEIILECV | 18 | A02 | 18 | + | + | | |
| E6 D32E, 134R/29-38 | TIHEIRLECV | 19 | A02 | 19 | + | 1 | | |
| E6 H85Y/81-90 | SEYRYYCYSL | 20 | A02 | 20 | + | + | | |
| E6 H85Y, L90V/81-90 | SEYRYYCYSV | 21 | A02 | 21 | + | + | | |
| E6 H85Y, L90V/83-90 | YRYYCYSV | 22 | A02 | 22 | + | + | | |
| E6 H85Y/84-93 | RYYCYSLYGT | 23 | A02 | 23 | + | + | | |
| E7/82-91 | LLMGTLGIVC | 24 | A02 | 24 | + | + | | |
| E7/82-92 | LLMGTLGIVCP | 25 | A02 | 25 | + | + | + | |
| E6 R171/9-17 | FQDPQERPI | 26 | A02 | 26 | + | | | |
| E6 R171/9-19 | FQDPQERPIKL | 27 | A02 | 27 | + | | | |
| E6/18-28 | KLPQLCTELQT | 28 | A02 | 28 | + | | | |
| E6/25-33 | ELQTTIHDI | 29 | A02 | 29 | + | | | |
| E6/28-38 | TTIHDIILECV | 30 | A02 | 30 | + | | + | |
| E6 H85Y/83-90 | YRYYCYSL | 31 | A02 | 31 | + | + | | |
| E7/7-17 | TLHEYMLDLQP | 32 | A02 | 32 | + | | | |
| E7/76-86 | IRTLEDLLMGT | 33 | A02 | 33 | + | | + | |

Table 1(iii): Peptides presented via HLA-A03/A11

| | | | | | | | |
|---|---|---|---|---|---|---|---|
| E6/37-46 | CVYCKQQLLR | 34 | A03 | 34 | + | | + |
| E6/37-46 | CVYCKQQLLR | 34 | A11 | 35 | * | | + |
| E6/33-41 | IILECVYCK | 35 | A11 | 36 | + | | + |
| E6/125-133 | HLDKKQRFH | 36 | A03 | 37 | + | | + |
| E7/89-97 | IVCPICSQK | 37 | A03 | 38 | + | | + |
| E7/89-97 | IVCPICSQK | 37 | A11 | 125 | +* | | + |
| E6 D32E/31-41 | HEIILECVYCK | 38 | A11 | 39 | + | + | |
| E6/52-62 | FAFRDLCIVYR | 39 | A11 | 40 | + | + | |
| E6/53-62 | AFRDLCIVYR | 40 | A11 | 41 | + | + | |
| E6/59-67 | IVYRDGNPY | 41 | A11 | 42 | + | + | |
| E6/65-75 | NPYAVCDKCLK | 42 | A11 | 43 | + | + | |
| E6/68-77 | AVCDKCLKFY | 43 | A03 | 44 | + | + | |
| E6/68-77 | AVCDKCLKFY | 43 | A11 | 126 | +* | + | |
| E6/68-78 | AVCDKCLKFYS | 44 | A11 | 45 | + | + | |
| E6/69-79 | VCDKCLKFYSK | 45 | A11 | 46 | + | + | |
| E7/88-97 | GIVCPICSQK | 46 | A03 | 47 | + | + | |
| E6/8-18 | MFQDPQERPRK | 47 | A03 | 48 | + | | |
| E6/67-77 | YAVCDKCLKFY | 48 | A11 | 49 | + | | |
| E6/68-75 | AVCDKCLK | 49 | A03 | 50 | + | | |
| E6/72-80 | KCLKFYSKI | 50 | A03 | 51 | + | | |
| E6/75-83 | KFYSKISEY | 51 | A03 | 52 | + | | |
| E6/75-84 | KFYSKISEYR | 52 | A03 | 53 | + | | |
| E6 L90V/84-94 | RHYCYSVYGTT | 53 | A03 | 54 | + | | |
| E6/93-101 | TTLEQQYNK | 54 | A03 | 55 | + | | |
| E6/105-115 | DLLIRCINCOK | 55 | A11 | 56 | + | + | |
| E6/107-115 | LIRCINCOK | 56 | A03 | 57 | + | | |
| E6/109-119 | RCINCQKPLCP | 57 | A03 | 58 | + | | |
| E6/129-138 | KQRFHNIRGR | 58 | A03 | 59 | + | | |
| E7/87-97 | LGIVCPICSQK | 59 | A11 | 60 | + | | |
| E6/92-101 | GTTLEQQYNK | 116 | A03 | 127 | + | | |
| E6/84-91 | RHYCYSLY | 117 | A03 | 128 | + | 1 | |

Table 1(iv): Peptides presented via HLA-A24

| | | | | | | | | |
|---|---|---|---|---|---|---|---|---|
| E6/38-45 | VYCKQQLL | 60 | A24 | 61 | − | | | + |
| E7/49-57 | RAHYNIVTF | 61 | A24 | 62 | − | | | + |
| E7/56-65 | TFCCKCDSTL | 62 | A24 | 63 | + | | + | |
| E6/66-76 | PYAVCDKCLKF | 63 | A24 | 64 | + | | | |
| E6 L90V/81-91 | SEYRHYCYSVY | 64 | A24 | 65 | + | + | | |
| E6 L90V/90-99 | VYGTTLEQQY | 65 | A24 | 66 | + | + | | |
| E7/47-57 | PDRAHYNIVTF | 66 | A24 | 67 | + | + | | |
| E7/48-57 | DRAHYNIVTF | 67 | A24 | 68 | + | + | | |
| E7/51-59 | HYNIVTFCC | 68 | A24 | 69 | + | + | | |
| E7 S63F/56-63 | TFCCKCDF | 69 | A24 | 70 | + | + | | |
| E6 A68G/60-69 | VYRDGNPYGV | 70 | A24 | 71 | + | | | |
| E6/75-83 | KFYSKISEY | 51 | A24 | 72 | + | | | |
| E6/85-95 | HYCYSLYGTTL | 71 | A24 | 73 | + | + | | |
| E6/88-95 | YSLYGTTL | 72 | A24 | 74 | + | | | |
| E6/125-135 | HLDKKQRFHNI | 73 | A24 | 75 | + | | | |
| E6/127-134 | DKKQRFHN | 74 | A24 | 76 | + | | | |
| E7/50-57 | AHYNIVTF | 75 | A24 | 77 | + | | | + |
| E7/51-58 | HYNIVTFC | 119 | A24 | 130 | + | | | |
| E7 S63F/56-65 | TFCCKCDFTL | 120 | A24 | 131 | + | | | |

Table 1(v): Peptides presented via HLA-B07

| | | | | | | | |
|---|---|---|---|---|---|---|---|
| E6/15-22 | RPRKLPQL | 76 | B07 | 78 | + | + | + |
| E7/5-13 | TPTLHEYML | 77 | B07 | 79 | * | | + |
| E7/49-57 | RAHYNIVTF | 61 | B07 | 80 | +* | | + |
| E6/15-25 | RPRKLPQLCTE | 78 | B07 | 81 | + | + | |
| E6/95-103 | LEQQYNKPL | 79 | B07 | 82 | + | + | |
| E6/134-144 | NIRGRWTGRCM | 80 | B07 | 83 | + | + | |
| E7/46-55 | EPDRAHYNIV | 81 | B07 | 84 | + | + | |
| E7/83-93 | LMGTLGIVCPI | 12 | B07 | 85 | + | + | |
| E6/15-24 | RPRKLPQLCT | 82 | B07 | 86 | + | | |
| E6/101-108 | KPLCDLLI | 83 | B07 | 87 | | | |
| E6/142-151 | RCMSCCRSSR | 84 | B07 | 88 | + | | |
| E6 R17G/15-22 | RPGKLPQL | 121 | B07 | 132 | | | + |
| E6 Q21D/15-24 | RPRKLPDLCT | 122 | B07 | 133 | + | + | |
| E6E120D/118-126 | CPDEKQRHL | 123 | B07 | 134 | + | | NA |
| E6/19-28 | LPQLCTELQT | 124 | B07 | 135 | + | | |

Table 1(vi): Peptides presented via HLA-B15

| | | | | | | | |
|---|---|---|---|---|---|---|---|
| E6/41-50 | KQQLLRREVY | 85 | B15 | 89 | + | + | + |
| E6/53-61 | AFRDLCIVY | 86 | B15 | 90 | + | + | + |
| E7/15-23 | LQPETTDLY | 87 | B15 | 91 | +* | + | + |
| E7/43-52 | GQAEPDRAHY | 88 | B15 | 92 | + | + | + |
| E6/42-50 | QQLLRREVY | 89 | B15 | 93 | + | | + |
| E7/49-57 | RAHYNIVTF | 61 | B15 | 94 | + | | + |
| E6 L90V/89-99 | SVYGTTLEQQY | 90 | B15 | 95 | − | | + |
| E7/15-24 | LQPETTDLYC | 91 | B15 | 96 | − | | + |
| E7/15-25 | LQPETTDLYCY | 92 | B15 | 97 | − | | + |
| E6/52-61 | FAFRDLCIVY | 93 | B15 | 98 | + | − | |
| E6/57-67 | LCIVYRDGNPY | 94 | B15 | 99 | + | − | |
| E6/68-77 | AVCDKCLKFY | 43 | B15 | 100 | + | + | |
| E6 L90V/81-91 | SEYRHYCYSVY | 64 | B15 | 101 | + | + | |
| E6 L90V/83-91 | YRHYCYSVY | 95 | B15 | 102 | + | + | |
| E6/44-54 | LLRREVYDFAF | 96 | B15 | 103 | + | | |
| E6/45-54 | LRREVYDFAF | 97 | B15 | 104 | + | | |
| E6/59-69 | IVYRDGNPYAV | 98 | B15 | 105 | + | | |
| E6/68-76 | AVCDKCLKF | 99 | B15 | 106 | + | | |
| E6/73-83 | CLKFYSKISEY | 100 | B15 | 107 | + | | |
| E6/76-84 | FYSKISEY | 101 | B15 | 108 | + | | |
| E6/77-86 | YSKISEYRHY | 102 | B15 | 109 | − | | |
| E6/78-86 | SKISEYRHY | 103 | B15 | 110 | − | | |
| E6/81-88 | SEYRHYCY | 104 | B15 | 111 | + | | |
| E6/81-91 | SEYRHYCYSLY | 9 | B15 | 112 | 1 | | |
| E6/97-106 | QQYNKPLCDL | 105 | B15 | 113 | + | | |
| E6/113-121 | CQKPLCPEE | 106 | B15 | 114 | + | | |
| E6/122-132 | KQRHLDKKQRF | 107 | B15 | 115 | + | | |
| E6/129-139 | KQRFHNIRGRW | 108 | B15 | 116 | + | + | |
| E6/134-144 | NIRGRWTGRCM | 80 | B15 | 117 | + | | |
| E7/7-15 | TLHEYMLDL | 109 | B15 | 118 | + | | |
| E7/43-51 | GQAEPDRAH | 110 | B15 | 119 | + | | |
| E7/82-90 | LLMGTLGIV | 111 | B15 | 120 | + | | |
| E7/50-57 | AHYNIVTF | 125 | B15 | 136 | | | + |
| E6/79-86 | KISEYRHY | 126 | B15 | 137 | + | | |
| E6/136-144 | RGRWTGRCM | 127 | B15 | 138 | + | | |
| E7/42-52 | AGQAEPDRAHY | 128 | B15 | 139 | + | | |
| E6/76-83 | FYSKISEY | 129 | B15 | 140 | + | | |

TABLE 2
Prediction vs. experimental confirmation of peptide functionality

| | | | num- |
|---|---|---|---|
| peptide type | number | peptide type | ber |
| possible binding peptides | approx. 9000 | | |
| predicted to bind to HLA A1, | 201 | | |
| A2, A3/A11, B07, or B15 | | | |
| experientally confirmed | 280 | thereof predicted | 99 |
| binders | | to bind | |
| | | thereof not predicted | 181 |
| | | to bind | |
| experimentally confirmed | 133 | thereof not predicted | 77 |
| immunogenic peptides | | | |
| experimentally confirmed | 34 | therof not predicted | 8 |
| presented peptides | | | |

### REFERENCES

Altman et al. (1996), Science 274:94
Bassani-Sternberg et al. (2016), Nat Commun, 7, 13404. doi:10.1038/ncomms13404
Becker & Riemer (2022), Frontiers Immunol, doi: 10.3389/fimmu.2022.883989
Blatnik (2018), Proteomics 18:1700390, DOI: 10.1002/pmic.201700390
Bonsack et al. (2019), Cancer Immunol Res; 7(5):719
Bourgault Villada et al., Clin Exp Immunol 2010; 159(1):45
Chardin & McCormick (1999), Cell 97, 153-155.
Chong et al. (2018), Mol Cell Proteomics, 17(3), 533-548. doi:10.1074/mcp.TIR117.000383
Currier et al. (2002), J Immunol Methods 260, 157-172.
Habib et al. (2022), Cells 6; 11(3):421. doi: 10.3390/cells11030421
Hara et al., Int J Oncol 2005; 27(5):1371
Jang et al., Cancer 2012; 118(8):2173
Kast et al., J Immunol 1994; 152(8):3904
Krishna et al. (2018) Cancer Res.2018; 78(21):6159
Lefranc et al. (2003), Dev. Comp. Immunol., 27:55
MacLean et al. (2010), Bioinformatics, 26(7), 966-968. doi:10.1093/bioinformatics/btq054
Mizuuchi et al., Exp Mol Pathol 2012; 92(1):185
Mollenhauer et al. (1990), Biochim Biophys Acta 1031, 225-246.
Ressing et al., J Immunol 1995; 154(11):5934
Riemer et al., J Biol Chem 2010; 285(38):29608
Salek et al. (2022), Analyt Bioanalyt Chem, 414(8), 2545-2552. doi:10.1007/s00216-022-03931-w
Sidney et al. (2008), BMC Immunology 9 Art. No. 1, doi.org/10.1186/1471-2172-9-1
Stanke et al. (2010), J Immunol Methods 360, 56-65.
Steinbach & Riemer (2018), Int J Cancer 142:224
Toprak et al. (2014), Mol Cell Proteomics, 13(8), 2056-2071. doi:10.1074/mcp.0113.036475
Tsang et al. (2017), Vaccine 35(19):2605
Ugel et al., Cancer Res. 2009; 69(24):9376

1. An in vitro method for producing an immunoreactive agent against human cancer cells infected with HPV16-related virus, said method comprising
expressing an at least partial nucleic acid sequence encoding an immunoreactive agent obtained from an immune cell stimulated by a complex comprising a human leukocyte antigen (HLA) and a stimulation peptide (stimulation complex), wherein said stimulation peptide
(i) consists of an amino acid sequence selected from SEQ ID NOs:1 and 2 and said HLA is from HLA supertype HLA-A01;
(ii) consists of an amino acid sequence of SEQ ID NO: 11 and said HLA is from HLA supertype HLA-A02,
(iii) consists of an amino acid sequence selected from SEQ ID NOs:34 to 37 and said HLA is from HLA supertype HLA-A03/A11;
(iv) consists of an amino acid sequence selected from SEQ ID NOs:60 and 61 and said HLA is from HLA supertype HLA-A24;
(v) consists of an amino acid sequence selected from SEQ ID NOs:61, 76, and 77 and said HLA is from HLA supertype HLA-B07; or
(vi) consists of an amino acid sequence selected from SEQ ID NOs:61 and 85 to 92 and said HLA is from HLA supertype HLA-B15;
and wherein an HPV16-derived peptide consisting of the same amino acid sequence as said stimulation peptide has been verified to be presented by human HPV16-positive cancer cells.

2. The method of claim 1, wherein said stimulation peptide has been verified to stimulate human anti-stimulation peptide immune cells when presented as a human leukocyte antigen (HLA)-complex.

3. The method of claim 1, wherein said stimulation complex is comprised on an antigen presenting cell (APC) or an artificial APC presenting said at least one stimulation complex or is an HLA oligomer comprising said stimulation peptide.

4. The method of claim 1, wherein said immune cells are T cells expressing a T cell receptor recognizing the stimulation complex and/or are B cells recognizing the stimulation complex.

5. The method of claim 1, wherein said T cells are CD4+ or CD8+ T cells, preferably are CD8+ T cells, preferably are CD8+ effector memory T cells.

6. The method of claim 1, wherein said HPV16-related virus is HPV16, HPV31, HPV33, HPV35, HPV52, HPV58, or HPV67, more preferably is HPV16.

7. A method for stimulating immune cells specifically binding to human host cells infected with a HPV16-related virus, said method comprising
(A) contacting a complex comprising a human leukocyte antigen (HLA) and a stimulation peptide (stimulation complex) with immune cells, and
(B) thereby stimulating immune cells specifically binding to said human host cells, wherein said stimulation peptide is selected from the list consisting of SEQ ID NOs:1, 2, 11, 34-37, 60, 61, 76, 77, and 85-92 and wherein an HPV16-derived peptide consisting of the same amino acid sequence as said stimulation peptide has been verified to be presented by human HPV16-positive cancer cells.

8. The method of claim 7, wherein said method is a method for stimulating and identifying immune cells specifically binding to said human host cells, and wherein said method further comprises (C) identifying stimulated immune cells stimulated in step (B).

9. The method of claim 7, wherein said method is a method for stimulating and enriching immune cells specifically binding to said human host cells and wherein said method further comprises step (D) enriching stimulated immune cells stimulated in step (B) and optionally identified in step (C).

10. The method of claim 8, wherein said method further comprises a step of contacting the immune cells of step (B) with a derivative of said stimulation complex carrying a detectable label and identifying and optionally enriching by use of said detectable label immune cells binding to said stimulation complex.

11. The method of claim 8, wherein (i) said method is an in vitro method or (ii) wherein step (A) comprises administering said stimulation complex to a subject, wherein said subject is a non-human animal and wherein said subject is sacrificed after step (C) or (D).

12. A method for determining the nucleic acid sequence of at least a part of an immunoreactive agent specifically binding to human host cells infected by a HPV16-related virus, said method comprising stimulating, identifying, and optionally enriching, immune cells specifically binding to said human host cells according to the method of claim 1, and the further step of sequencing at least a part of at least one polynucleotide encoding said immunoreactive agent in said immune cell.

13. (canceled)

14. (canceled)

15. The method of claim 1, wherein said immunoreactive agent is a TCR, an anti-immune complex antibody or a fragment or derivative thereof.

16. The method of claim 11, wherein the immunoreactive agent is a TCR, an anti-immune complex antibody, or a fragment or derivative thereof.
